(19) United States
(12) Patent Application Publication
(10) Pub. No.: US 2017/0348430 A1
(43) Pub. Date: Dec. 7, 2017

(54) MICELAR DELIVERY SYSTEM BASED ON ENZYME-RESPONSIVE AMPHIPHILIC PEG-DENDRON HYBRID

(71) Applicant: RAMOT AT TEL AVIV UNlVERSITY LTD., Tel Aviv (IL)

(72) Inventors: Roey Jacob AMIR, Tel Aviv (IL); Marina BUZHOR, Tel Aviv (IL); Assaf Josef HARNOY, Tel Aviv (IL); Ido ROSENBAUM, Tel Aviv (IL); Liat FRID, Tel Aviv (IL)

(73) Assignee: RAMOT AT TEL AVIV UNLVERSITY LTD., Tel Aviv (IL)

(21) Appl. No.: 15/509,962

(22) PCT Filed: Feb. 25, 2015
(86) PCT No.: PCT/IL2015/050212

§ 371 Date: Mar. 9, 2017

Related U.S. Application Data
(60) Provisional application No. 62/047,697, filed on Sep. 9, 2014.

(51) Int. Cl. A61K 47/69; A61K 47/60

(52) U.S. Cl. CPC A61K 47/6907; A61K 47/60

(57) ABSTRACT

The present invention relates to an enzymatic stimuli-responsive amphiphilic hybrid delivery system in micellar form, based on a hydrophilic polyethylene glycol (PEG) polymer conjugated to a hydrophobic dendron. The delivery system disassembles upon enzymatic stimuli/cleavage. The present invention further provides methods of use of the hybrid delivery system and to a kit comprising the same.

### FIELD OF THE INVENTION

The present invention relates to an enzymatic stimuli-responsive amphiphilic hybrid delivery system in micellar form, based on a hydrophilic polyethylene glycol (PEG) polymer conjugated to a hydrophobic dendron. The delivery system disassembles upon enzymatic stimuli/cleavage. The present invention further provides methods of using the hybrid delivery system and to a kit comprising the same.

### BACKGROUND OF THE INVENTION

Stimuli-responsive micelles that can disassemble and release their encapsulated cargo upon external stimuli have gained increasing attention in the past years. Their potential utilization as nanocarriers has gained relevance in prophylaxis and therapeutics as drug delivery, in food industry, cosmetic, agrochemicals and textile fabrics. These responsive materials are inspired by the ability of many supramolecular assemblies in nature to alter their structures and activity in response to changes in their environment. Thus, mimicking these systems via synthetic approaches is of increasing interest. The current approaches for developing such novel stimuli-responsive polymers are based on response to changes in pH, temperature, irradiated light, redox potential or their combination. While these approaches offer great control over the triggering of the disassembly processes, substantial advantages could be achieved by utilizing enzymes as stimuli. Enzymes are attractive and unique stimuli with great potential, as they are highly substrate specific and propagate an amplified response via catalytic reactions. As many diseases are characterized by imbalances in the expression and activity of specific enzymes in the diseased tissue, this overexpression could potentially be translated into the selective activation of advanced drug delivery platforms.

However, up to date, there are only limited reports of enzyme responsive synthetic micellar nanostructures, most of them are based on breaking an amphiphilic block copolymer into a soluble hydrophilic polymer and an insoluble hydrophobic block.

Azagarsamy et al., 2009, J. Am. Chem. Soc. 131: 14184-14185 describes dendrimer-based amphiphilic assemblies that can noncovalently sequester hydrophobic guest molecules and release these guests in response to an enzymatic trigger. This is achieved by incorporating enzyme sensitive functionalities at the lipophilic face of the dendrons. This feature causes a change in the hydrophilic-lipophilic balance (HLB) when the enzyme is encountered, effecting disassembly and guest-molecule release. The reported structures have a particle size between 100-200 nm prior to disassembly.

Ku et al., 2011, J. Am. Chem. Soc. 133: 8392-8395, studied the reversible switchable morphology of micellar nanoparticles with enzymes. The micelles are based on amphiphilic polymer-peptide block copolymer containing substrates for four different cancer-associated enzymes: protein kinase A, protein phosphatase-1, and matrix-metalloproteinases 2 and 9. Upon enzymatic cleavage a variety of morphologies of polymeric amphiphilic aggregates are formed.

Rao et al., 2013, J. Am. Chem. Soc. 135: 14056-14059 describes an amphiphilic diblock copolymer comprising PEG and polystyrene wherein an azobenzene linkage is incorporated at the junction of the two polymers. Upon cleavage of the azo-based linkage, the polystyrene fragment precipitates out of the solution and the hydrophilic PEG remains solubilized.

Rao et al. 2014, J. Am. Chem. Soc. 136, 5872-5875 describes a system comprising poly(styrene) and an enzyme-sensitive methacrylate-based polymer segment carrying azobenzene side chains. The azobenzene linkages cleave upon enzymatic activation, triggering a series of reactions that transforms the hydrophobic methacrylate polymer into a hydrophilic hydroxyethyl methacrylate structure. This leads the polymer to self-assemble into a micellar nanostructure in water.

Amir et al., 2009, J. Am. Chem. Soc. 131: 13949-13951, describes enzymatic activation of a water soluble diblock copolymer to obtain an amphiphilic diblock copolymer, which self-assembles into colloidal nanostructures.

Amir et al., 2003, Angew. Chem. Int. Ed. 42: 4494-4499, describes self-immolative dendrimers, wherein a self-immolative chain fragmentation is initiated with a single cleavage of a trigger moiety at the dendritic core. This event leads to a spontaneous release of all the tail units of the dendrimer. This technology is also described in US Patent Application No. 2005/0271615, to some of the inventors of the present invention.

Gillies et al., 2004, J. Am. Chem. Soc. 126: 11936-11943 discloses a linear-dendritic block copolymers comprising poly(ethylene oxide) and either a polylysine or a polyester dendrimer wherein hydrophobic groups are attached to the dendrimer periphery by acid-sensitive cyclic acetal linkages. These copolymers form stable micelles in aqueous solution at neutral pH but disintegrate into unimers at mildly acidic pH.

de Groot et al., 2003, Angew. Chem. Int. Ed. 42: 4490-4494 discloses cascade-release dendrimers based on monomeric multiple-release building blocks. Following a single activation step at the dendritic core, a cascade of self-elimination reactions is triggered, which induces release of all the end groups attached at the dendrimer periphery.

Harnoy, A S et al., 2014, J Am Chem Soc. 136(21): 7531-4 disclose enzyme responsive amphiphilic PEG-dendron hybrids and their assembly into micellar nanocarriers.

There is an ongoing and unmet need in the art to develop a delivery system that can initiate a controlled release of ligands upon enzymatic activation. Ideally, such system should be highly modular for various delivery applications, easily prepared/produced, highly selective and should remain soluble subsequent to the release of the ligands.

### SUMMARY OF THE INVENTION

The present invention relates to an amphiphilic hybrid delivery system in micellar form, based on a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group. The present invention further provides methods of use thereof for different applications including biomedical, cosmetic, and textile among others and to a kit comprising the same.

The present invention is based on modular methodology for the synthesis of polymer-dendron hybrids as stimuli responsive delivery systems. Conjugation of enzymatically cleavable groups (“innocent” or “active”) to the end groups of the dendrimer allows unprecedented control over the degree of loading and release of the active ingredient (e.g., drugs, diagnostic agents, etc.). Furthermore, the novel molecular architecture allows harnessing its highly defined structure and amphiphilic nature in order to form polymeric carriers that can self-assemble into “smart” micellar assemblies. These stimuli-responsive micelles are expected to disassemble and release their cargo upon enzymatic cleavage of the covalent bonds between the dendron and the hydrophobic end-groups. In some embodiments, such “smart” assemblies can be further utilized to encapsulate active ingredients that cannot be conjugated to the polymer due to the lack of available functional groups on the active ingredient.

In one aspect, the present invention is based on the modular design of enzyme responsive amphiphilic hybrids composed of linear PEG and a stimuli responsive dendron with enzyme cleavable hydrophobic end-groups. These amphiphilic PEG-dendron hybrids self-assemble in water into micelles with a hydrophilic PEG shell and a hydrophobic core, which potentially can be utilized to encapsulate hydrophobic cargo molecules. In the presence of the activating enzyme, the hydrophobic end groups can be cleaved from the dendron, making it more hydrophilic. This change in amphiphilicity results in destabilization of the micellar aggregates, leading to their disassembly and release of soluble PEG-dendron hybrids and their encapsulated cargo (FIG. 1). The unique morphology of the micelles, with a highly packed PEG shell gives the micelle protecting properties such as avoidance of nonspecific activation with other proteins/proteases and leaching diminution of the encapsulated ligands.

The amphiphilic hybrid delivery systems of the invention are particularly advantageous as they self-assemble into thermodynamically stable micelles having a well-controlled disassembly profile. The superiority of the modular design is manifested by efficient and simple synthesis as well as complete control of the loading capacity of the hydrophobic end groups as well as the encapsulation of additional cargo molecules within the micelle. The modularity of these PEG-dendron hybrids allows control over the disassembly rate of the formed micelles by simply tuning the PEG length. Such smart amphiphilic hybrids could potentially be applied for the fabrication of nanocarriers with adjustable release rates for delivery applications. The spherical nanocarriers disclosed herein possess beneficial structural and physical attributes including well-defined molecular and supermoleculare structure, monodispersity, specific size, thermodynamic stability, encapsulation ability, and water solubility. As the released polymer-dendron is highly hydrophilic, it can be easily washed away after the delivery of the active cargo. In addition, these delivery platforms do not require the use of additional surfactants or surface-active materials in order to solubilize hydrophobic compounds as the hybrid structures function as macromolecular surfactants.

The present invention is also based in part on the unexpected finding that the disassembly of the micelle and release rates of the active ingredients can be adjusted by rational tuning of structural parameters of the nanoparticles (such as hydrophilicity and length of the linear polymer, dendron generation, number of cleavable moieties, linkage chemistry and polymer/dendron weight ratio) as well as the stimuli cleavable moiety parameters (i.e., enzyme specificity, amount of enzyme, incubation time, etc.).

Thus, according to one aspect, the present invention provides an amphiphilic hybrid delivery system in micellar form, comprising a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group.

In another aspect, the present invention provides amphiphilic hybrid delivery system in micellar form, comprising a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group; and wherein the hydrophobic end group is conjugated to the dendron through an enzymatically cleavable functional group selected from the group consisting of an ester, a carbamate, a carbonate, a urea, a sulfate, an amidine, an ether, a phosphate, a phosphoamide, sulfamates, and a trithionate.

In yet another aspect, the present invention provides amphiphilic hybrid delivery system in micellar form, comprising a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group; and wherein the hydrophobic end group is or is derived from an agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

In some embodiments, the micelle has an average particle size of less than about 100 nm, preferably about 50 nm or lower, more preferably about 10 nm to 50 nm, and most preferably about 10 nm to 20 nm. Each possibility represents as separate embodiment of the present invention.

According to some embodiments, the dendron comprises a plurality of enzymatically cleavable hydrophobic end groups.

According to some embodiments, the enzymatically cleavable hydrophobic end group is present at one or more of the terminal repeating units (i.e., terminal generations) of the hydrophobic dendron, and/or in intermediary generations of the dendron. In other embodiments, the enzymatically cleavable hydrophobic end group is present only at the terminal repeating units of the hydrophobic dendron (i.e., the enzymatically cleavable hydrophobic end group is not present in intermediary generations of the dendron).

According to some embodiments, the hydrophobic dendron comprises a first generation which is covalently bound to the PEG polymer, directly or through a linker moiety/branching unit, and comprises at least one functional group capable of binding to a further generation or to said enzymatically cleavable hydrophobic end group; and optionally, at least one additional generation which is covalently bound to said first generation or preceding generation and optionally to a further generation, wherein each of said optional generations comprises at least one functional group capable of binding to said first generation, to a preceding generation, to a further generation, and/or to said enzymatically cleavable hydrophobic end group, each of said bonds being formed directly or through a linker or branching unit.

According to certain embodiments, each generation of the hydrophobic dendron comprises a linear or branched C1-C20 alkylene, C2-C20 alkenylene, C2-C20 alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, NH—C(═O)—O—, —S(═O)—, —S(═O)—O—, PO(═O)—O—, and any combination thereof. Each possibility represents as separate embodiment of the present invention.

According to other embodiments, each generation of the dendron is derived from a compound selected from the group consisting of HX—CH₂—CH₂—XH, HX—(CH₂)₁₋₃—CO₂H, and HX—CH₂—CH(XH)—CH₂—XH wherein X is independently at each occurrence NH, S or O. In one currently preferred embodiment, the dendron is derived from a compound selected from the group consisting of HS—CH₂—CH₂—OH, HS—(CH₂)₁₋₃—CO₂H and HS—CH₂—CH(OH)—CH₂—OH. Each possibility represents as separate embodiment of the present invention.

The hydrophobic dendron of the present invention comprises a preferred number of generations in the range of 0 to 5, more preferably 0 to 3. In one embodiment, the hydrophobic dendron is a generation 0 (GO) dendron. In another embodiment, the hydrophobic dendron is a generation 1 (G1) dendron. In another embodiment, the hydrophobic dendron is a generation 2 (G2) dendron. In yet another embodiment, the hydrophobic dendron is a generation 3 (G3) dendron.

According to some embodiments, the PEG has an average molecular weight between about 0.5 and 40 kDa, e.g., 2 kDa, 5 kDa and 10 kDa. Preferably, the PEG has at least 10 repeating units of ethylene glycol monomers.

According to some embodiments, the hybrid delivery system further comprises a linker moiety and/or a branching unit which connects the PEG polymer to the first generation dendron, and/or forms a part of the first generation, and/or connects between dendron generations. In one embodiment, the linker moiety and/or the branching unit is selected from a group consisting of a substituted or unsubstituted acyclic, cyclic or aromatic hydrocarbon moiety, heterocyclic moiety, a heteroaromatic moiety or any combination thereof. Each possibility represents as separate embodiment of the present invention. In one currently preferred embodiment, the linker moiety/branching unit is a substituted arylene which may be positioned between the PEG and the first generation or may form a part of the first generation, or alternatively may be positioned at one or more intermediary generations of the dendron. The branching unit may in some cases impart functionality (e.g., UV absorbance or other desired properties). Each possibility represents a separate embodiment of the present invention.

According to various embodiments, each of the linker moiety/branching unit may be connected to the PEG or to other dendron generations through a functional group selected from the group consisting of —O—, —S—, —NH—, —C(═O)—, —C(═O)—O—, —OC(═O)—O—, —C(═O)—NH—, —NH—C(═O)—NH—, NH—C(═O)—O—, —S(═O)—, —S(═O)—O—, PO(═O)—O—, —C═C—, —≡C—, —(CH₂)ₜ— wherein t is an integer of 1-10, and any combination thereof. One representative example of a functional group linking the PEG to the dendron is —S—(CH₂)ₜ—NHC(O)—. Each possibility represents as separate embodiment of the present invention.

According to some embodiments, the enzymatically cleavable hydrophobic end group is conjugated to the dendron through an enzymatically cleavable functional group selected from the group consisting of an ester, an amide, a carbamate, a carbonate, a urea, a sulfate, an amidine, an ether, a phosphate, a phosphoamide, sulfamates, and a trithionate. Each possibility represents as separate embodiment of the present invention.

According to some embodiments, the enzymatically cleavable hydrophobic end group is conjugated to the dendron through an amide which is cleavable by an amidase. In one embodiment, the amidase is selected form the group of aryl-acylamidase, aminoacylase, alkylamidase, and phthalyl amidase. Each possibility represents as separate embodiment of the present invention.

According to some embodiments, the enzymatically cleavable hydrophobic end group is conjugated to the dendron through an ester which is cleavable by an esterase. In one embodiment, the esterase is selected from the group consisting of carboxylesterase, arylesterase, and acetylesterase. Each possibility represents as separate embodiment of the present invention.

According to other embodiments, the enzymatically cleavable hydrophobic end group is cleaved by an enzyme which is (i) present in greater amount at; or (ii) produced in greater quantity at, or (iii) has higher activity in cells near or at a site of disease or infection. Each possibility represents as separate embodiment of the present invention.

The enzymatically cleavable hydrophobic end group may be an “innocent” group, i.e., it is biologically inactive. Alternatively, the enzymatically cleavable hydrophobic end group may itself be, or may be derived from a biologically or diagnostically active agent which is released upon disassembly of the micelle. In either case (i.e., delivery systems containing “innocent” or “active hydrophobic end groups), the hybrid delivery system may further comprise a biologically or diagnostically active compound encapsulated (non-covalently) within the micelle, wherein the active compound is released upon disassembly of the micelle. Each possibility represents a separate embodiment of the present invention.

In some embodiments, the hydrophobic end group which is covalently attached to the dendron and the compound which is encapsulated within the micelle are the same, and they are both biologically/diagnostically active compounds, or they are derived therefrom. In another embodiment, the hydrophobic end group which is covalently attached to the dendron and the active compound which is encapsulated within the micelle are different, and they are both biologically/diagnostically active compounds, or they are derived therefrom. In other embodiment, the hydrophobic end group which is covalently attached to the dendron is biologically inactive, and the micelle non-covalently encapsulates a biologically/diagnostically active compound which is released upon disassembly of the micelle.

The hydrophobic end group which is attached/conjugated to the dendron, and/or the compound which is encapsulated within the micelle may each independently be a biologically or diagnostically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody. In specific embodiment, these compounds selected from the group consisting of an anti-proliferative agent, a nonsteroidal anti-inflammatory agent, an antibiotic agent, an antimicrobial agent, an anti-viral agent, an immunosuppressant agent, an immunomodulator agent, an anti-hypertensive agent, a chemosensitizing agent, an anti-histamine agent, a general anesthetic agent, a local anesthetic agent, an analgesic agent, an anti-fungal agent, a vitamin, a fat-soluble vitamin, an hypnotic agent, a sedative agent, an anxiolytic agent, an antidepressant agent, an anticonvulsant agent, a narcotic analgesic agent, a narcotic antagonist agent, an anticholinesterase agent, a sympathomimetic agent, a parasympathomimetic agent, a ganglionic stimulating agent, a ganglionic blocking agent, an antimuscarinic agent, an adrenergic blocking agent, an autacoid and autacoid antagonist, digitalis and digitalis congeners, diuretic and saliuretic agents, a cholesterol lowering agent, an antineoplastic agent, hemoglobin and hemoglobin derivatives and polymer, a hormonal agent, a hormonal antagonist agent, and combination thereof. Each possibility represents as separate embodiment of the present invention.

In a preferred embodiment, the hydrophobic end group which is attached/conjugated to the dendron, and the compound which is encapsulated within the micelle are each independently selected from the group consisting of coumarin, methyl salicylate, aspirin, ibuprofen, naproxen, famciclovir, valacyclovir, acyclovir, penicillin-V, azlocillin, tetracycline, daunorubicin, doxorubicin, anthracycline, mitomycin C, aminopterin, mycophenolate mofetil, azathioprine, sirolimus, glucocorticoid, methotrexate, azathioprine, ciclosporin, tacrolimus, thalidomide, lenalidomide, pomalidomide, chlorothiazide, metolazone, amiloride, acrivastine, bilastine, buclizine, cimetidine, clobenpropit, desflurane, isoflurane, sevoflurane, propofol, methohexital, benzocaine, dibucaine, lidocaineproparacaine, paracetamol, morphine, oxycodone, celecoxib, flupirtine, amphotericin B, candicidin, bifonazole, butoconazole, fluconazole, abafungin, anidulafungin, retinol, thiamine, riboflavin, biotin, ergocalciferol, retinal, retinol, amobarbital, alprazolam, zopiclone, midazolam, amobarbital, alprazolam, sertraline, clobazam, codeine, naltrexone, physostigmine, ephedrine, dimethylphenylpiperazinium, pentamine, atropine, terazosin, histamine, hydrochlorothiazide, statin, tibolone, ganirelix acetate, septrin and derivatives thereof. Each possibility represents as separate embodiment of the present invention.

According to some embodiments, the hybrid delivery system is represented by the structure of formula (I), which is provided in the Detailed Description hereinbelow. Specific examples of the hybrid delivery system of formula (I) are described in the Detailed Description hereinbelow.

In another aspect, the present invention provides a method of delivering the amphiphilic hybrid system comprising the step of contacting the amphiphilic hybrid delivery system with an enzyme to induce cleavage of the enzymatically cleavable hydrophobic end group, thereby disassembling the micelle.

In another aspect, the present invention provides a kit for delivering the amphiphilic hybrid system comprising in one compartment the amphiphilic hybrid system and in a second compartment an enzyme capable of cleaving the enzymatically cleavable hydrophobic end group so as to disassemble the micelle.

The present invention will be more fully understood from the following figures and detailed description of the preferred embodiments thereof.

### BRIEF DESCRIPTION OF THE DRAWINGS

FIG. 1: Schematic representation of the self-assembly and disassembly of the micellar nanocarrier.

FIG. 2A-2C: Schematic representation of PEG-dendron hybrids, 1a-c and their micellar assemblies (left); Size of the micelles by DLS at a hybrids concentration of 160 μM before (t=0, no enzyme) and after (t=8h or 4h, with enzyme) the addition of the activating enzyme.

FIG. 3A-3C: TEM micrographs of micelles formed from PEG-dendron hybrids 1a-c.

FIG. 4: Fluorescence spectra of Nile red (1.25 μM) in the presence of PEG-dendron hybrid 1a (160 μM) shows the decrease in fluorescence intensity upon the addition of the activating enzyme, PGA (0.14 μM).

FIG. 5: Fluorescence spectra of Nile red (1.25 μM) in the presence of PEG-dendron hybrid 1b (160 μM) shows the decrease in fluorescence intensity upon the addition of the activating enzyme, PGA (0.14 μM).

FIG. 6: Fluorescence spectra of Nile red (1.25 μM) in the presence of PEG-dendron hybrid 1c (160 μM) shows the decrease in fluorescence intensity upon the addition of the activating enzyme, PGA (0.14 μM).

FIG. 7: Fluorescence emission intensity spectra of compound 1b in the presence of 0.66 μM of Esterase from porcine liver (PLE enzyme).

FIG. 8: Fluorescence emission intensity spectra of compound 1b in the absence of the activating enzyme PGA after 12 hour in buffer.

FIG. 9: Fluorescence emission intensity of compound 6b is unaffected in the presence of 1.4 μM PGA enzyme.

FIG. 10: HPLC monitoring of micelle degradation in the presence of 0.14 μM PGA enzyme for compound 1b over time.

FIG. 11: HPLC monitoring of micelle degradation in the presence of 1.4 μM PGA enzyme for compound 1b over time.

FIG. 12: Change in fluorescence intensity and HPLC analysis of the enzymatic degradation of the PEG-dendron hybrid 1a (160 μM with 0.14 μM PGA enzyme). Partially degraded intermediates are shown schematically.

FIG. 13: Change in fluorescence intensity and HPLC analysis of the enzymatic degradation of the PEG-dendron hybrid 1b (160 μM with 0.14 μM PGA enzyme). Partially degraded intermediates are shown schematically.

FIG. 14: Change in fluorescence intensity and HPLC analysis of the enzymatic degradation of the PEG-dendron hybrid 1c (160 μM with 0.14 μM PGA enzyme). Partially degraded intermediates are shown schematically.

FIG. 15: HPLC monitoring of compound 1b in the presence of 0.66 μM PLE enzyme after 3 hours. No degradation was observed showing the specificity of the PGA enzyme.

FIG. 16: Comparison of the disassembly rates (fluorescence assay) of micelles formed by PEG-dendron hybrids 1a-c.

FIG. 17: Esterase-responsive cleavage of the PEG-dendron hybrid 11b.

FIG. 18: DLS measurements of the amphiphilic PEG-dendron hybrid 11b before (solid diamond) and after (open square) the addition of the activating enzyme.

FIG. 19: ¹H-NMR spectra of compound 11b in D₂O showing only PEG protons in the absence of the enzyme (A); After the addition of the activating enzyme, the dendron becomes hydrophilic and its protons reappear in the spectrum (B).

FIG. 20: Fluorescence emission intensity spectra overlay of compound 11b (160 μM) with 0.23 μM PLE.

FIG. 21: Fluorescence emission intensity spectra overlay of compound 15b (40 μM) with 8.5 μM PLE.

FIG. 22: HPLC monitoring of micelle degradation in the presence of 0.23 μM PLE enzyme for compound 11b over time.

FIG. 23: HPLC monitoring of micelle degradation in the presence of 8.5 μM PLE enzyme for compound 15b over time.

FIG. 24: HPLC analysis of the enzymatic degradation of the PEG-dendron hybrid 11b (160 μM with 0.23 μM PLE enzyme).

FIG. 25: HPLC analysis of the enzymatic degradation of the PEG-dendron hybrid 15b (40 μM with 8.5 μM PLE enzyme).

FIG. 26: Change in fluorescence intensity and HPLC analysis of the enzymatic degradation of the PEG-dendron hybrid 11b and micelles disassembly.

FIG. 27: Release of encapsulated dyes from enzyme responsive micelle 11b.

FIG. 28: Release of bound dyes from enzyme responsive micelle 15b.

FIG. 29: Chemical structures of several hybrid delivery systems according to the invention.

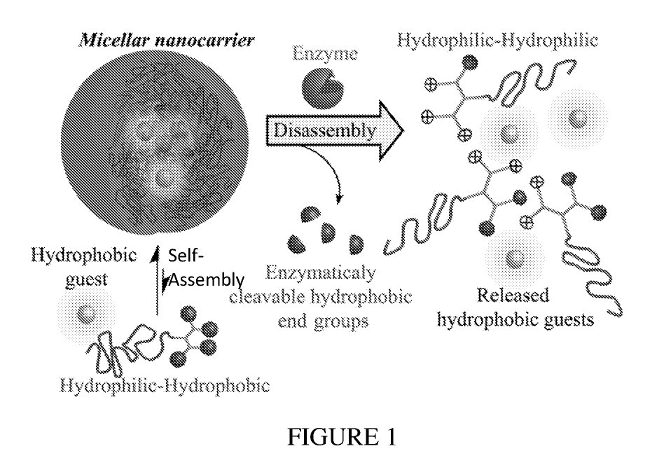

### DETAILED DESCRIPTION OF THE PRESENT INVENTION

The Amphiphilic Hybrid Delivery System

According to one aspect, the present invention provides an amphiphilic hybrid delivery system in micellar form, comprising a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group. In one embodiment, the hydrophobic end group is conjugated to the dendron through an enzymatically cleavable functional group selected from the group consisting of ester, a carbonate, a carbamate, a urea, a sulfate, an amidine, an ether, a phosphate, a phosphoamide, sulfamates, and a trithionate. In another embodiment, the hydrophobic end group is or is derived from an agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

In some embodiments, the micelle has an average particle size of less than about 100 nm, preferably about 50 nm or lower, more preferably about 10 nm to 50 nm, and most preferably about 10 nm to 20 nm. Each possibility represents as separate embodiment of the present invention.

A “dendron” is a hyper-branched monodisperse organic molecule defined by a tree-like or generational structure. In general, dendrons possess three distinguishing architectural features: a linker moiety; an interior area containing generations with radial connectivity to the linker moiety; and a surface region (peripheral region) of terminal moieties. According to certain embodiments, each generation of the hydrophobic dendron comprises a linear or branched C1-C20 alkylene, C2-C20 alkenylene, C2-C20 alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, NH—C(═O)—O—, —S(═O)—, —S(═O)—O—, PO(═O)—O—, and any combination thereof. Each possibility represents as separate embodiment of the present invention. According to some embodiments, the hybrid delivery system further comprises a linker moiety and/or a branching unit which connects the PEG polymer to the first generation dendron, and/or forms a part of the first generation, and/or connects between dendron generations. In one embodiment, the linker moiety and/or the branching unit is selected from a group consisting of a substituted or unsubstituted acyclic, cyclic or aromatic hydrocarbon moiety, heterocyclic moiety, a heteroaromatic moiety or any combination thereof. Each possibility represents as separate embodiment of the present invention. Specific examples of linker moieties/branching units useful for this invention include but are not limited to, arylenes, which may be substituted with one or more hydroxyls (e.g., phenols), trimethylolpropane, glycerine, pentaerythritol, polyhydroxy phenols such as phloroglucinol, propylene glycol, tri-substituted alkylamines, diethylenetriamine, triethylenetetramine, diethanolamine, triethanolamine, amino carboxylic acids, such as ethylenediaminetetraacetic (EDTA) and porphyrin, ethylene glycol, ethylenediamine di-substituted alkylamines, diethylenetriamine, triethylenetetramine, diethanolamine, fumaric, maleic, phthalic, malic acid, 6-aminohexanol, 6-mercaptohexanol, 10-hydroxydecanoic acid, 1,6-hexanediol, beta-alanine, 2-aminoethanol, 2-aminoethanethiol, 5-aminopentanoic acid, and 6-aminohexanoic acid among others. Each possibility represents as separate embodiment of the present invention. In one currently preferred embodiment, the linker moiety/branching is an unsubstituted or substituted arylene or phenol which may be positioned between the PEG and the first generation or may form a part of the first generation, or alternatively may be positioned at one or more intermediary generations of the dendron. The linker/branching unit may further provide additional functionality to the hybrid delivery system (e.g., UV absorption). According to various embodiments, each of the linker moiety/branching unit may be connected to the PEG or to other dendron generations through a functional group selected from the group consisting of —O—, —S—, —NH—, —C(═O)—, —C(═O)—O—, —OC(═O)—O—, —C(═O)—NH—, —NH—C(═O)—NH—, NH—C(═O)—O—, —S(═O)—, —S(═O)—O—, PO(═O)—O—, —C═C—, —≡C—, —(CH₂)ₜ— wherein t is an integer of 1-10, and any combination thereof. One representative example of a functional group linking the PEG to the dendron is —S—(CH₂)ₜ—NHC(O)—. Each possibility represents as separate embodiment of the present invention.

The hydrophilic PEG polymer is a currently preferred polymer to prepare the block co-polymer hybrid of the present invention as it is generally recognized as safe for use in food, cosmetics, medicines and many other applications by the US Food and Drug Administration. PEG has beneficial physical and/or chemical properties such as water-solubility, non-toxic, odorless, lubricating, nonvolatile, and non-intrusive which are particularly suitable for pharmaceutical utility.

There are many commercial available derivatives of PEG, all of which may be useful in the present invention, such as but not limited to methoxy PEG (mPEG), amine-terminated PEG (PEG-NH₂), acetylated PEG (PEG-Ac) carboxylated PEG (PEG-COOH), thiol-terminated PEG (PEG-SH), N-hydroxysuccinimide-activated PEG (PEG-NHS), NH₂—PEG-NH₂ or NH₂—PEG-COOH. Each possibility represents as separate embodiment of the present invention. These PEG derivatives may be subjected to further chemical modifications and substitutions.

According to some embodiments, the PEG has an average molecular weight between about 0.5 and 40 kDa. In one currently preferred embodiment, the hydrophilic PEG polymer is an mPEG. In another currently preferred embodiment, the PEG polymer has a molecular weight of about 2 kDa. In another currently preferred embodiment, the PEG polymer has a molecular weight of about 5 kDa. In yet another currently preferred embodiment, the PEG polymer has a molecular weight of about kDa.

According to some embodiments, the hybrid delivery system is represented by the structure of formula (I):

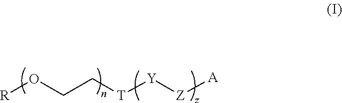

wherein

R is H or a C1-C4 alkylene group;

T is absent or is a functional group selected from the group consisting of —O—, —S—, —NH—, —C(═O)—, —O—C(═O)—O—, —C(═O)—O—, —C(═O)—NH—, —NH—C(═O)—NH—, NH—C(═O)—O—, —S(═O)—, —S(═O)—O—, PO(═O)—O—, —C═C—, —≡C—, —(CH₂)ₜ— wherein t is an integer of 1-10, and any combination thereof.

Y is independently at each occurrence absent or is a linker moiety/branching unit;

Z is independently at each occurrence a dendron repeating unit selected from the group consisting of:

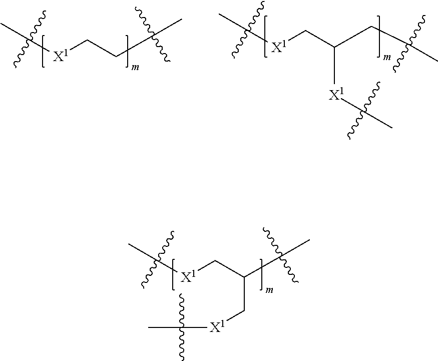

and any combination of the foregoing;

wherein X¹ is independently, at each occurrence, selected from the group consisting of a O, S and NH;

A is a hydrophobic end group which is conjugated to the dendron through an enzymatically cleavable functional group selected from the group consisting of an ester, an amide, a carbamate, a carbonate, a urea, a sulfate, an amidine, an ether, a phosphate, a phosphoamide, sulfamates, and a trithionate;

n is an integer in the range of 1 to 1,500; and

m and z are each an integer of 1 to 15.

In some embodiments, n is an integer in the range of 1 to 1,000.

In some embodiments, the hydrophobic end group A is or is derived from a biologically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

According to other embodiments, the terminal repeating unit of said dendron is represented by any of the following structures:

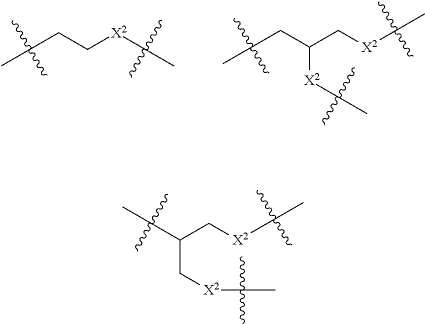

wherein X² has the same meaning as X¹.

According to yet other embodiments, the hydrophobic end group A is conjugated to the dendron through a functional group represented by the structure:

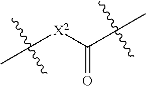

wherein X² is a part of the terminal repeating unit of said dendron and C(═O) is part of hydrophobic end group; or wherein X² is part of the hydrophobic end group and C(═O) is a part of the terminal repeating unit of said dendron, or wherein X²—C(═O) are part of the hydrophobic end group, or wherein X²—C(═O) is part of the terminal repeating unit of said dendron; and wherein X² has the same meaning as X¹.

Specific examples of the hybrid delivery system of formula (I) include, but are not limited to, any one or more of the following structures:

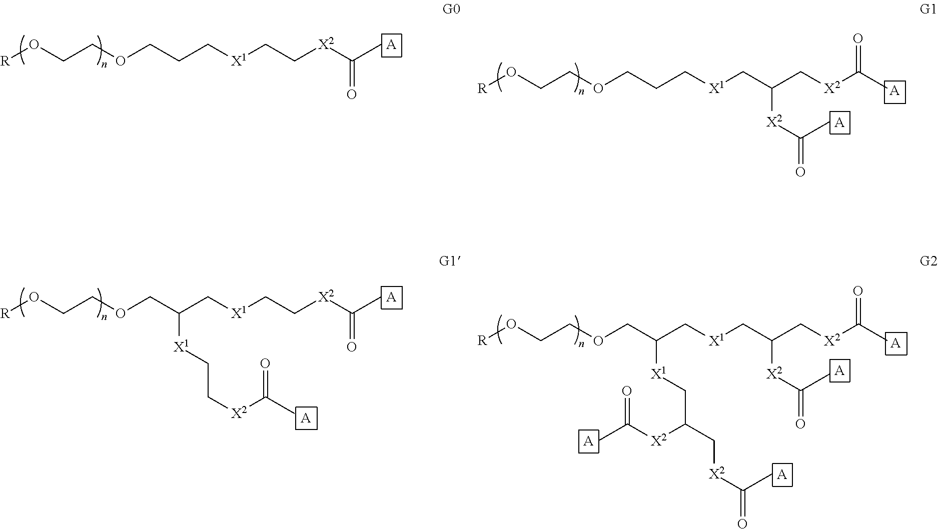

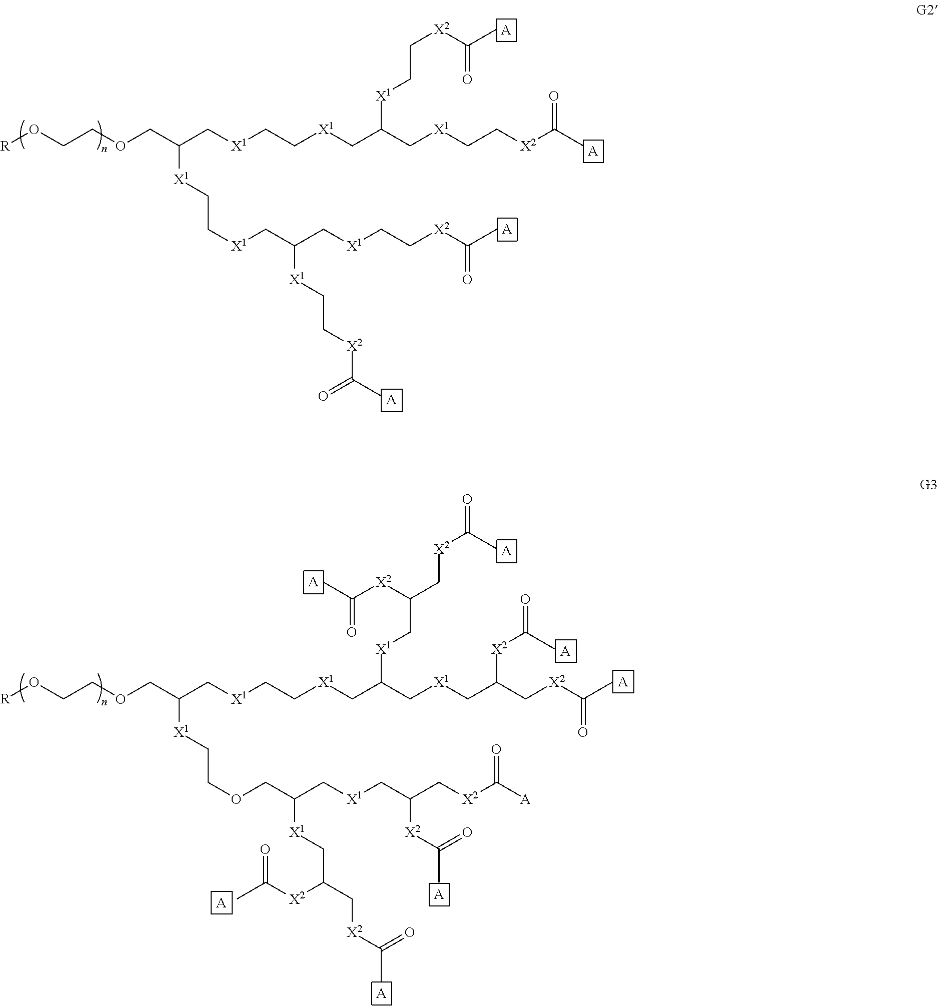

wherein each X¹ and X² is independently at each occurrence selected from the group consisting of O, S and NH;
R is H or a C1-C4 alkylene group;
A, alone or together with C(═O) is a hydrophobic end group; and
n is an integer of 1 to 1,500.
In some embodiments, n is an integer in the range of 1 to 1,000.

Each possibility represents as separate embodiment of the present invention.

Additional specific example of the hybrid delivery system of formula (I) include the following structure:

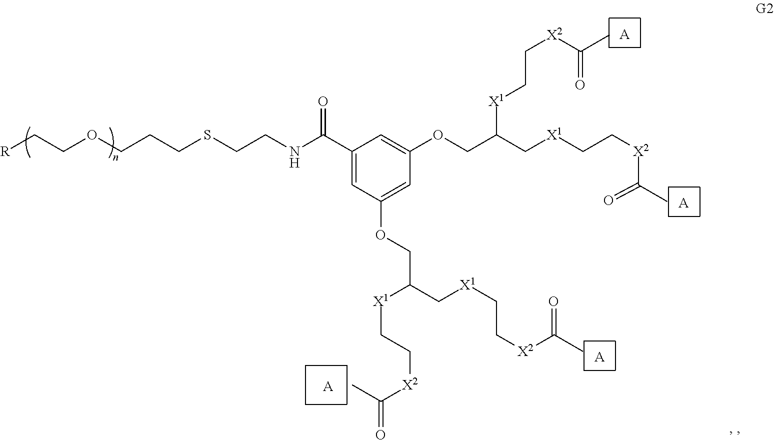

wherein each X¹ and X² is independently at each occurrence selected from the group consisting of O, S and NH;
R is H or an C1-C4 alkylene group;
A, alone or together with C(═O) is a hydrophobic end group; and
n is an integer of 1 to 1,500, preferably 1 to 1,000.

Also contemplated are analogues of compounds of formulae GO, G1, G2, G2′, G2″ and G3 wherein the linkage of A to —X²—C(═O)— is reversed, i.e., the compounds incorporate the following moiety:

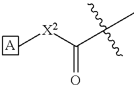

wherein X² is part of the hydrophobic end group A or part of the dendron.

In some embodiments, the hybrid delivery system is represented by the following structures which are depicted in the experimental section below: 1a-1c (1a: 2 kDa PEG; 1b: 5 kDa PEG; 1c: 10 kDa PEG); 11a-11c (11a: 2 kDa PEG; 11b: 5 kDa PEG; 11c: 10 kDa PEG); and 15a-15c (15a: 2 kDa PEG; 15b: 5 kDa PEG; 15c: 10 kDa PEG). Additional specific example of the hybrid delivery system of formula (I) are those depicted in FIG. 29. It is understood by a person of skill in the art that the phenylacetamide group, i.e., the hydrophobic end group in the compounds exemplified in FIG. 29, can be replaced with any other ligand, including biologically and diagnostically active ligands as described herein. Such additional compounds are also encompassed by the present invention.

According to some embodiments, the enzymatically cleavable hydrophobic end group is conjugated to the dendron through an enzymatically cleavable functional group is selected from the group consisting of an ester, an amide, a carbamate, a carbonate, a urea, a sulfate, an amidine, an ether, a phosphate, a phosphoamide, sulfamates, and a trithionate. Each possibility represents as separate embodiment of the present invention.

According to some embodiments, the enzymatically cleavable hydrophobic end group is conjugated to the dendron through an amide which is cleavable by an amidase. In one embodiment, the amidase is selected form the group of aryl-acylamidase, aminoacylase, alkylamidase, and phthalyl amidase. Each possibility represents as separate embodiment of the present invention.

According to some embodiments, the enzymatically cleavable hydrophobic end group is conjugated to the dendron through an ester which is cleavable by an esterase. In one embodiment, the esterase is selected from the group consisting of carboxylesterase, arylesterase, and acetylesterase. Each possibility represents as separate embodiment of the present invention.

According to other embodiments, the enzymatically cleavable hydrophobic end group is cleaved by an enzyme which is (i) present in greater amount at; or (ii) produced in greater quantity at, or (iii) has higher activity in cells near or at a site of disease or infection. Each possibility represents as separate embodiment of the present invention.

The modular design of the hybrid delivery systems of the present invention provides control over the disassembly of the micelle and release rate of the hydrophobic end groups and/or encapsulated cargo. This can be achieved by adjusting structural features of the nanocarriers (such as length of PEG polymer, dendron generation, number of enzymatically cleavable moieties, linkage chemistry and polymer/dendron weight ratio) as well as enzymatic-tuning parameters (e.g., enzyme specificity, amount of enzyme and incubation time). An example for the effect of the length of PEG polymer on the disassembly rates of the micelles is shown in FIG. 16.

Hydrophobic End Groups and Encapsulated Compounds

The enzymatically cleavable hydrophobic end group “A” may be an “innocent” group, i.e., it is not biologically active. Alternatively, the enzymatically cleavable hydrophobic end group may itself be, or may be derived from a biologically or diagnostically active agent. Each possibility represents a separate embodiment of the present invention. It is understood that the biologically or diagnostically active agent, or the biologically inactive group is released from the micelle upon enzymatic cleavage

Also, the delivery system of the present invention may further contain a biologically or diagnostically active compound encapsulated (non-covalently) within the micelle, wherein the active compound is released upon disassembly of said micelle.

According to some embodiments, the hydrophobic end group which is attached to the dendron and the compound which is encapsulated within the micelle are the same compound, or they are derived from the same compound. In other embodiments, the hydrophobic end group which is attached to the dendron and the compound which is encapsulated within the micelle are different compounds. One embodiment of the present invention encompasses micelles which contain hydrophobic end groups that are not in themselves biologically active, wherein the micelle encapsulates (non-covalently) an active ingredient and releases it upon cleavable of the hydrophobic end groups. In an alternative embodiment, the hydrophobic end group is or is derived from an active ingredient (e.g., biologically or diagnostically active ingredient). The micelle formed therefrom releases the active ingredient upon enzymatic cleavage of the hydrophobic end group. In yet another embodiment, the hydrophobic end group is or is derived from an active ingredient (e.g., biologically or diagnostically active ingredient), and in addition the micelle encapsulates (non-covalently) an active ingredient and releases is upon cleavage of the hydrophobic end group. The active ingredient which is part of the hydrophobic end group and which is encapsulated within the micelle may be the same or different, with each possibility representing a separate embodiment of the present invention.

In some non-limiting embodiments, the hydrophobic end group which is attached/conjugated to the dendron, and the compound which is encapsulated within the micelle are each independently a biologically or diagnostically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent, and an antibody. Each possibility represents as separate embodiment of the present invention.

In addition to list of agents given above, the hybrid delivery system is suited for use in a variety of applications where a specific delivery of material/cargo is desired.

A pharmaceutically active agent refers to a chemical or biological molecule having therapeutic, diagnostic or prophylactic effects in vivo. In a specific embodiment, the pharmaceutically active agent is selected from the group consisting of an anti-proliferative agent, a nonsteroidal anti-inflammatory agent, an antibiotic agent, an antimicrobial agent, an anti-viral agent, an immunosuppressant agent, an immunomodulator agent, an anti-hypertensive agent, a chemosensitizing agent, an anti-histamine agent, a general anesthetic agent, a local anesthetic agent, an analgesic agent, an anti-fungal agent, a vitamin, a fat-soluble vitamin, an hypnotic agent, a sedative agent, an anxiolytic agent, an antidepressant agent, an anticonvulsant agent, a narcotic analgesic agent, a narcotic antagonist agent, an anticholinesterase agent, a sympathomimetic agent, a parasympathomimetic agent, a ganglionic stimulating agent, a ganglionic blocking agent, an antimuscarinic agent, an adrenergic blocking agent, an autacoid and autacoid antagonist, digitalis and digitalis congeners, diuretic and saliuretic agents, a cholesterol lowering agent, an antineoplastic agent, hemoglobin and hemoglobin derivatives and polymer, a hormonal agent, a hormonal antagonist agent, and combination thereof. Each possibility represents a separate embodiment of the invention.

Non-limiting examples of pharmaceutically active agents that are useful in the present invention include: anti-proliferative agent (e.g., aminopterin, mycophenolate mofetil, azathioprine, and sirolimus), anti-inflammatory agent (e.g., coumarin, celecoxib, methyl salicylate, aspirin, ibuprofen, and naproxen), antiviral agent (e.g., famciclovir, valacyclovir, and acyclovir), antibiotics (e.g., penicillin-V, azlocillin, and tetracyclines), an antimicrobial agent (e.g., septrin, cefazolin, and aminopenicillin), chemotherapeutic agent (e.g., daunorubicin, doxorubicin, N-(5,5-diacetoxypentyl)doxorubicin, anthracycline, mitomycin C, mitomycin A, 9-amino camptothecin, aminopterin, actinomycin, N8-acetyl spermidine, 1-(2-chloroethyl)-1,2-dimethanesulfonyl hydrazine, bleomycin, tallysomycin, etoposide, camptothecin, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine, and vinblastine), immunosuppressant agent (e.g., glucocorticoid, methotrexate, azathioprine, ciclosporin, tacrolimus, sirolimus, infliximab, etanercept, oradalimumab, basiliximab, and daclizumab), an immunomodulator agent (e.g., thalidomide, lenalidomide, and pomalidomide), an anti-hypertensive agent (e.g., chlorothiazide, chlorthalidone, metolazone, amiloride, and triamterene), anti-hystamin agent (e.g., buclizine, cimetidine, and clobenpropit), general anesthetic agent (e.g., desflurane, isoflurane, sevoflurane, propofol, and methohexital), local anesthetic agent (e.g., benzocaine, butamben, dibucaine, lidocaine, oxybuprocaine, pramoxine, proparac aine, proxymetacaine, and tetracaine), an analgesic agent (e.g., paracetamol, salicylate, morphine, oxycodone, aspirin, and flupirtine), an anti-fungal agent (e.g., butoconazole, albaconazole, fluconazole, abafungin, amorolfine, and anidulafungin), a vitamin (e.g., retinol, thiamine, riboflavin, biotin, ascorbic acid, tocopherol, and phylloquinone), a fat-soluble vitamin (e.g., retinal, cholecalciferol, ergocalciferol, tocopherol, tocotrienol, and phylloquinone), a hypnotic agent (e.g., amobarbital, pentobarbital, secobarbital, cloroqualone, etaqualone, alprazolam, lorazepam, diazepam, clonazepam, zopiclone, and eszopiclone), a sedative agent (e.g., midazolam and amobarbital), an anxiolytic agent (e.g., alprazolam and etizolam), an antidepressant agent (e.g., sertraline, citalopram, and fluoxetine), an anticonvulsant agent (e.g., clobazam, oxcarbazepine, and tiagabine), a narcotic analgesic agent (e.g., morphine and codeine), a narcotic antagonist agent (e.g., naloxone and naltrexone), an anticholinesterase agent (e.g., physostigmine and neostigmine), a sympathomimetic agent (e.g., ephedrine, pseudoephedrine, and amphetamine), a ganglionic stimulating agent (e.g., nicotine, lobeline, and dimethylphenylpiperazinium), a ganglionic blocking agent (e.g., hexamethonium, chlorisondamine, and pentamine), an antimuscarinic agent (e.g., atropine, and scopolamine), an adrenergic blocking agent (e.g., terazosin, prazosin, and propranolol), an autacoid agent (histamine) and autacoid antagonist, diuretic and saliuretic agents (e.g., furosemide, hydrochlorothiazide, and ethacrynic acid), a cholesterol lowering agent (e.g., statins, niacin, ezetimibe, and clofibrate), an antineoplastic agent (e.g., cyclophosphamide, and chlormethine), a hormonal agent (e.g., andarine, danazol, and tibolone), a hormonal antagonist agent (e.g., cetrorelix, and ganirelix acetate) and derivatives thereof, among others. Each possibility represents a separate embodiment of the invention.

A cosmetic active agent refers to a chemical or biological molecule having restorative, cleansing, protective, moisturizing, toning, conditioning or soothing effects, on skin, hair, or nails. Such cosmetic active agents may advantageously be included in various beauty care products including for example, day creams, night creams, makeup-removing creams, foundation creams, antisun creams, fluid foundations, makeup-removing milks, protective or body care milks, after-sun milks, skincare lotions, gels, mousses, cleansing lotions, antisun lotions, artificial tanning lotions, bath compositions, deodorizing compositions, aftershave gels and lotions and hair-removing creams. Each possibility represents as separate embodiment of the present invention.

An anti-oxidant refers to a chemical or biological molecule having anti-oxidant effects. Anti-oxidants include for example, butylated hydroxytoluene (BHT), butylated hydroxy anisol (BHA) and carnosic acid, among others. Each possibility represents a separate embodiment of the invention.

A preservative refers to a chemical or biological molecule having inhibitory effects against microorganisms, including bacteria, viruses, fungi and molds. Preservatives include for example, methyl paraben, ethyl paraben, propyl paraben and butyl paraben, among others. Each possibility represents a separate embodiment of the invention.

A colorant refers to a chemical or biological molecule having pigmenting effects. Examples of colorants that are suitable for the present invention include, for example, pigments, dyes and the like.

A food additive refers to a chemical or biological molecule which is added to a processed food product. Food additives include for example, vitamins, preservatives, anti-oxidants, flavouring agents, among others. Each possibility represents a separate embodiment of the invention.

An imaging agent refers to a chemical or biological molecule used to diagnose a disease, track disease progression and monitoring treatment effects. Imaging molecules include, but are not limited to, Gadolinium, ⁶⁴Cu-ATSM, ¹⁸F-fluoride, FLT, FDG, FMISO, Gallium, Thallium, Barium, FITC, tryptophan, rhodamine, 4′,6-diamidino-2-phenylindole (DAPI), fluorescein and it's derivatives, red dyes, green dyes such as AlexaFlor and fluorescent proteins such as GFP/eGFP, and YFP, among others. Each possibility represents a separate embodiment of the invention.

A diagnostic agent refers to a chemical or biological molecule used to identify a disease, disorder or medical condition as well as monitor treatment effects. Diagnostic agents include radiopharmaceuticals, contrast agents for use in imaging techniques, allergen extracts, activated charcoal, different testing strips (e.g., cholesterol, ethanol, and glucose), pregnancy test, breath test with urea ¹³C, and various stains/markers. Each possibility represents as separate embodiment of the present invention.

A fragrance refers to a chemical or biological molecule which produces an olfactory effect. Fragrances include perfume oils such as natural aroma mixtures, such as those accessible from plant sources, for example pine, citrus, jasmine, patchouli, rose, or ylang-ylang oil. Also suitable are muscatel, salvia oil, chamomile oil, clove oil, lemon balm oil, mint oil, peppermint oil, spearmint oil, cinnamon leaf oil, linden blossom oil, juniper berry oil, vetiver oil, olibanum oil, galbanum oil, and labdanum oil, as well as orange blossom oil, neroli oil, orange peel oil, and sandalwood oil. Other suitable fragrances include but are not limited to fruits such as almond, apple, cherry, grape, pear, pineapple, orange, strawberry, raspberry; musk, flower scents such as lavender-like, rose-like, iris-like, and carnation-like. Other pleasant scents include herbal scents such as rosemary, thyme, and sage; and woodland scents derived from pine, spruce and other forest smells. Each possibility represents a separate embodiment of the invention. A list of suitable fragrances is provided in U.S. Pat. Nos. 4,534,891, 5,112,688 and 5,145,842, the contents of which are hereby incorporated by reference.

The term “derived from” as used herein means a moiety that is derived from an active compound (i.e., any of the biologically or diagnostically active compounds described herein) and that is incorporated into the hybrid systems of the present invention. A derivative of an active moiety may be formed, e.g., by removing one or more of the atoms of said compound or adding one or more atoms or functional groups so as to chemically conjugate it to the dendron.

### Chemical Definitions

The term “C1-C4/C1-C20 alkylene” used herein alone or as part of another group denotes a bivalent radicals of 1 to 4/20 carbons, which is bonded at two positions connecting together two separate additional groups (e.g., CH₂). Examples of alkylene groups include, but are not limited to —(CH₂)—, (CH₂)₂, (CH₂)₃, (CH₂)₄, etc.

The term “C2-C20 alkenylene” denotes a bivalent radical of 2 to 20 carbons which contains at least one double bond, which is bonded at two positions connecting together two separate additional groups (e.g., —CH═CH—).

The term “C2-C20 alkynylene” denotes a bivalent radicals of 2 to 20 carbons containing at least one triple bond, which is bonded at two positions connecting together two separate additional groups (e.g., —C≡C—).

The term “arylene” denotes a bivalent radicals of aryl, which is bonded at two positions connecting together two separate additional groups.

The term “acyclic hydrocarbon” used herein denotes to any linear or branched, saturated and mono or polyunsaturated carbon atoms chain, or the residue of such compound after it has chemically bonded to another molecule. Preferred are acyclic hydrocarbon moieties containing from 1 to 20 carbon atoms. The acyclic hydrocarbon of the present invention may comprise one or more of an alkyl, an alkenyl, and an alkynyl moieties. Examples of acyclic hydrocarbon include, but are not limited to, n-propyl, isopropyl, n-butyl, sec-butyl, isobutyl and tert-butyl, n-pentyl, n-hexyl, vinyl, allyl, butenyl, pentenyl, propargyl, butynyl, pentynyl, and hexynyl. Each possibility represents as separate embodiment of the present invention.

The term “cyclic hydrocarbon” generally refers to a C3 to C8 cycloalkyl or cycloalkenyl which includes monocyclic or polycyclic groups. Non-limiting examples of cycloalkyl groups are cyclopropyl, cyclobutyl, cyclopentyl, cyclohexyl or cycloheptyl. The cycloalkyl group can be unsubstituted or substituted with any one or more of the substituents defined above for alkyl.

The term “aromatic hydrocarbon” used herein denotes to an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. Each possibility represents as separate embodiment of the present invention. The aryl group can be unsubstituted or substituted through available carbon atoms with one or more groups defined hereinabove for alkyl.

The terms “heterocyclic” or “heterocyclyl” used herein alone denote a five-membered to eight-membered rings that have 1 to 4 heteroatoms, such as oxygen, sulfur and/or nitrogen. These five-membered to eight-membered rings can be saturated, fully unsaturated or partially unsaturated. Preferred heterocyclic rings include piperidinyl, pyrrolidinyl, pyrrolinyl, pyrazolinyl, pyrazolidinyl, piperidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, and the like. Each possibility represents as separate embodiment of the present invention. The heterocyclyl group can be unsubstituted or substituted through available atoms with one or more groups defined hereinabove for alkyl.

The term “heteroaryl” used herein denotes a heteroaromatic system containing at least one heteroatom ring atom selected from nitrogen, sulfur and oxygen. The heteroaryl generally contains 5 or more ring atoms. The heteroaryl group can be monocyclic, bicyclic, tricyclic and the like. Also included in this expression are the benzoheterocyclic rings. If nitrogen is a ring atom, the present invention also contemplates the N-oxides of the nitrogen containing heteroaryls. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. Each possibility represents as separate embodiment of the present invention. The heteroaryl group may optionally be substituted through available atoms with one or more groups defined hereinabove for alkyl.

Any of the moieties described herein (e.g., alkeylene, alkenylene, alkynylene, arylene, acyclic and cyclic hydrocarbons, heterocyclic and heteroaromatic moieties) may be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryl, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, C₁ to C₄ alkylthio, arylthio, or C₁ to C₄ alkylsulfonyl groups. Any substituent can be unsubstituted or further substituted with any one of these aforementioned substituents. Each possibility represents as separate embodiment of the present invention.

All stereoisomers, optical and geometrical isomers of the compounds of the instant invention are contemplated, either in admixture or in pure or substantially pure form. The compounds of the present invention can have asymmetric centers at any of the atoms. Consequently, the compounds can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to phosphate, dihydrogen phosphate, hydrogen phosphate and phosphonate salts, and include salts formed with organic and inorganic anions and cations. Furthermore, the term includes salts that form by standard acid-base reactions of basic groups and organic or inorganic acids. Such acids include hydrochloric, hydrofluoric, hydrobromic, trifluoroacetic, sulfuric, phosphoric, acetic, succinic, citric, lactic, maleic, fumaric, cholic, pamoic, mucic, D-camphoric, phthalic, tartaric, salicyclic, methanesulfonic, benzenesulfonic, p-toluenesulfonic, sorbic, picric, benzoic, cinnamic, and like acids. Additional salts of the conjugates described herein may be prepared by reacting the parent molecule with a suitable base, e.g., NaOH or KOH to yield the corresponding alkali metal salts, e.g., the sodium or potassium salts. Additional basic addition salts include ammonium salts (NH₄⁺), substituted ammonium salts, Li, Ca, Mg, salts, and the like.

Uses

In another aspect, the present invention provides a method of delivering the amphiphilic hybrid system comprising the step of contacting the amphiphilic hybrid delivery system with an enzyme to induce cleavage of the enzymatically cleavable hydrophobic end group, thereby disassembling the micelle.

As used herein, the term “contacting” refers to bringing in contact with the amphiphilic hybrid delivery system of the present invention. Contacting can be accomplished to cells or tissue cultures, or to living organisms, for example humans. In one embodiment, the present invention encompasses contacting the amphiphilic hybrid delivery system of the present invention with a human subject.

As used herein, the term “contacting the amphiphilic hybrid delivery system” may be ex-vivo on a surface, on a device, in cell/tissue culture dish, in food and water, as well as in-vivo, among others. Alternatively, the contact may be in the body of a human or non-human subject.

Kits

In another aspect, the present invention provides a kit for delivering the amphiphilic hybrid system comprising in one compartment the amphiphilic hybrid system, and in a second compartment an enzyme capable of cleaving the enzymatically cleavable hydrophobic end group so as to disassemble the micelle.

The kit may further include appropriate buffers and reagents known in the art for administering/contacting the compartments listed above to a host cell or a host organism. The amphiphilic hybrid delivery system and the enzyme may be provided in solution and/or in lyophilized form. When the enzyme is in a lyophilized form, the kit may optionally contain a sterile and physiologically acceptable reconstitution medium such as water, saline, buffered saline, and the like.

According to some embodiments, associated with such compartments may be various written materials such as instructions for use.

The examples hereinbelow are presented in order to more fully illustrate some embodiments of the invention. They should, in no way be construed, however, as limiting the broad scope of the invention. One skilled in the art may readily devise many variations and modifications of the principles disclosed herein without departing from the scope of the invention.

### Examples

### Example 1

### Materials and Methods

Materials: Poly (Ethylene Glycol) methyl ether (2 kDa, 5 kDa and 10 kDa), 2-30 (Boc-amino)-ethanethiol (97%), 2,2-dimethoxy-2-phenylacetophenone (DMPA, 99%), Penicillin G Amidase from Escherichia coli (PGA), Esterase from porcine liver (PLE), Allyl bromide (99%), 4-Nitrophenol (99.5%), N,N′-dicyclohexylcarbodiimide (DCC, 99%), Sephadex® LH20 and dry DMF were purchased from Sigma-Aldrich. Cystamine hydrochloride (98%), potassium hydroxide and DIPEA were purchased from Merck. Trifluoroacetic acid (TFA) was purchased from Alfa Aesar and phenyl acetic acid was purchased from Fluka. Silica Gel 60 Å, 0.040-0.063 mm, sodium hydroxide and all solvents were purchased from Bio-Lab and were used as received. All solvents are HPLC grade. Deuterated solvents for NMR were purchased from Cambridge Isotope Laboratories, Inc.

Instrumentation: HPLC: All measurements were recorded on a Waters Alliance e2695 separations module equipped with a Waters 2998 photodiode array detector. ¹H and ¹³C NMR: spectra were recorded on Bruker Avance I and Avance III 400 MHz spectrometers. GPC: All measurements were recorded on Viscotek GPCmax by Malvern using refractive index detector. Infrared spectra: All measurements were recorded on a Bruker Tensor 27 equipped with a platinum ATR diamond. Fluorescence spectra: All measurements were recorded on an Agilent Technologies Cary Eclipse Fluorescence Spectrometer using quartz cuvettes. CMC: All measurements were recorded on a TECAN Infinite M200Pro device. MALDI-TOF MS: Analysis was conducted on a Bruker AutoFlex MALDI-TOF MS and also on a Waters MALDI synapt. DHB matrix was used. TEM: Images were taken by a Philips Tecnai F20 TEM at 200 kV. DLS: All measurements were recorded on a Malvern Zetasizer NanoZS.

Methods

### NMR

Chemical shifts are reported in ppm and referenced to the solvent. The molecular weights of the PEG-dendron hybrids were determined by comparison of the areas of the peaks corresponding to the PEG block (3.63 ppm) and the protons peaks of the dendrons.

### Gel Permeation Chromatography (GPC)

### Instrument Method:

### Columns: 2×PSS GRAM 1000 Å+PSS GRAM 30 Å

### Columns Temperature: 50° C.

Flow rate: 0.5 ml/min
Mobile phase: DMF+50 mM LiBr
Detector: Refractive index detector at 50° C.

### Injection Volume: 50 μL

General sample preparation: PEG product was dissolved in mobile phase to give a final concentration of 10 g/ml. Solution was filtered through a 0.22 μm PTFE syringe filter.
PEG standards (purchased from Sigma-Aldrich) were used for calibration.

### Critical Micelle Concentration (CMC) Measurements

### Instrument Method:

### Excitation: 550 nm

Emission intensity scan: 580-800 nm
General procedure: Into 100 ml PBS solution (pH 7.4), 45 μL of Nile red stock solution (0.88 mg/ml in Ethanol) was added and mixed to give a final concentration of 1.25 μM. Then, the amphiphilic hybrids of the present invention were dissolved directly into aqueous buffer solution (PBS, pH 7.4). Each solution was sonicated for 15 minutes and then filtered through a 0.22 μm nylon syringe filter. This solution was repeatedly diluted by a factor of 2 with diluent. 100 μL of each solution were loaded onto a 96 wells plate. The fluorescence emission intensity was scanned for each well. Maximum emission intensity was plotted vs. concentration in order to determine the CMC.

### Dynamic Light Scattering (DLS) Measurements

General sample preparation: The amphiphilic hybrids of the invention were dissolved in PBS buffer (pH 7.4) to give a final concentration of 160 μM. Each solution was sonicated for 15 minutes and filtered through a 0.22 μm nylon syringe filter. 800 μL of each solution was accurately transferred into a polystyrene cuvette and a measurement was performed (t=0).
Micelle degradation in the presence of 0.14 μM PGA enzyme: 0.8 μL of PGA enzyme stock solution (140 μM in PBS buffer pH 7.4) was added to 800 μL of each PEG-dendron hybrid solution (160 μM). Repeating measurements were performed every 2 hours.
Micelle degradation in the presence of 1.4 μM PGA enzyme: 8 μL of PGA enzyme stock solution (140 μM in PBS buffer pH 7.4) was added to 800 μL of each PEG-dendron hybrid solution (160 μM). Repeating measurements were performed every 3 minutes.

### Transmission Electron Microscopy (TEM) Measurements

General sample preparation: 5 mL sample solution was dropped cast onto carbon coated copper grids and inspected in a transmission electron microscope (TEM), operated at 200 kV (Philips Tecnai F20). The excessive solvent of the droplet was wiped away using a solvent-absorbing filter paper after 1 min and the sample grids were left to dry in air at room temperature for 5 minutes. This procedure was repeated three times. After the third cycle the sample grids were left to dry in air at room temperature overnight.

### Nile Red Fluorescence Measurements

### Instrument Method:

### Excitation: 550 nm

Emission scan: 575-800 nm
Excitation and Emission slits width: 20 nm
Scan rate: 600 nm/min
General Sample Preparation and Measurement: The amphiphilic hybrids of the invention were dissolved in PBS buffer (pH 7.4) to give a concentration of 160 μM. Each solution was sonicated for 15 minutes and then filtered through a 0.22 μm nylon syringe filter. 2.0 mL of this solution were accurately transferred to a quartz cuvette and 0.9 μL of Nile Red stock solution (0.88 mg/mL in Ethanol) was added to give a final concentration of 1.25 μM. A fluorescence emission scan was performed (t=0) and then 2.0 μL of PGA enzyme stock solution (140 μM in PBS buffer pH7.4) was added to give a final concentration of 0.14 μM. Repeating fluorescence scans were performed every 15 minutes for 20 hours.
Enzymatic Degradation with HPLC
Instrument method:

### Column: Phenomenex, Luna, C1 - - - 8, 150×4.6 mm, 5 μm.

### Column Temperature: 30° C.

Mobile Phase: Solution A: 0.1% TFA in H₂O:Acetonitrile 95:5 V/V. Solution B: 0.1% TFA in H₂O:Acetonitrile 5:95 V/V.

### Gradient Program:

| Time | % Sol. | % Sol. |
|---|---|---|
| [min] | A | B |
| 0 | 100 | 0 |
| 20.0 | 0 | 100 |
| 23.0 | 0 | 100 |
| 23.1 | 100 | 0 |
| 30.0 | 100 | 0 |

Injection volume: 20 μL.
Detector: UV at 295 nm, 2 Hz detection rate.
Needle Wash: 0.1% concentrated H₃PO₄ in MeOH.
Seal wash solution: H₂O:MeOH 90:10 V/V.
Diluent: PBS buffer pH7.4.
General sample preparation and Measurement: The hybrids of the invention were dissolved in diluent to give a concentration of 160 μM. Each solution was sonicated for 15 minutes and then filtered through a 0.22 μm nylon syringe filter. Then, 20 μL of each solution was injected to the HPLC as t=0 injection. Stock solution of the enzyme (PGA or PLE) in PBS (pH 7.4) was added and mixed manually. Enzymatic degradation was monitored at several time points by repeating 20 μL injections from the same solution over time.

### Example 2

Synthesis Protocol of the Amphiphilic PEG-Dendron Hybrids (1a-1c)

In the above scheme, MeO-PEG-NH₂ (compounds 2a-2c) is represented by the structure shown in Scheme 2.

The amphiphilic hybrids (1a-c) of the invention may be prepared by the process described in general Scheme 1 hereinabove. Briefly, the hybrid block copolymers were synthesized utilizing mono-methyl ether PEG-amine, 2a-c, as starting materials. Conjugation with an active ester of 3,5-bis(prop-2-yn-1-yloxy)benzoic acid, 3, yielded PEG-di-yne, 4a-c. The latter were further modified by thiol-yne reaction with N-Boc cysteamine, 5, to give tetra-functionalized PEG-dendrons, 6a-c, followed by deprotection of the Boc to yield PEG-tetra-amine, 7a-c. In the last step of the synthesis, 4-nitrophenyl ester of phenyl acetic acid, 8, was used to introduce the enzyme cleavable hydrophobic surface-groups. PEG-dendron hybrids, 1a-c, were obtained as off-white solids with overall yields of 76%, 86% and 93%, respectively.

The synthesized polymers and hybrids were characterized by ¹H and ¹³C-NMR, GPC, IR and MALDI in order to confirm their structures.

### General Procedure for MeO-PEG-Allyl Compounds 2a-c

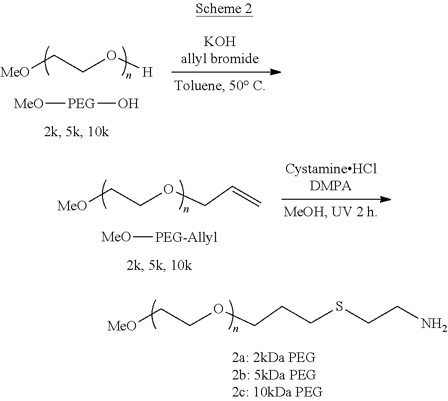

MeO-PEG-Allyl precursors may be prepared by the process described in general Scheme 2 hereinabove. Poly (ethylene glycol) methyl ether was dissolved in toluene (10 mL per 1 g) with KOH (10 eq.). The solution was refluxed for at least 1 hour using a Dean Stark water separation system. Solution was cooled down to 50° C. and then allyl bromide (10 eq.) was added slowly and the reaction was stirred overnight. The solution was filtered hot through celite, the celite was then washed with DCM. Solvents were evaporated in vacuum and the residue was re-dissolved in DCM (5 mL per 1 g PEG). MeO-PEG-Allyl product was precipitated by the dropwise addition of 1:1 v/v Ether:Hexane mixture (50 mL per 1 g PEG). Precipitate was filtered and washed with ether and then with hexane. The final white solid product was dried under high vacuum.

MeO-PEG2 kDa-Allyl: 3.00 g (1.5 mmol) Poly (ethylene glycol) methyl ether (Mₙ=2 kDa) were reacted according to the general procedure (I) and the product was obtained as a white solid (2.42 g) 80% yield. ¹H-NMR (CDCl₃): δ 5.85-5.95 (m, 1H, vinyl —CH═CH₂), 5.26 (dd, J=1.4 Hz, 17.2 Hz, 1H, trans vinyl —CH═CH₂), 5.17 (dd, J=1.0 Hz, 10.4 Hz, 1H, cis vinyl —CH═CH2), 4.01 (d, J=5.7 Hz, 2H, —O—CH₂—CH═CH₂), 3.44-3.82 (m, 206H, PEG backbone), 3.37 (s, 3H, H₃C—O—); ¹³C-NMR (CDCl₃) δ 134.9, 117.2, 72.4, 72.1, 70.7, 69.6, 59.1; FT-IR, v(cm⁻¹) 2878, 1466, 1456, 1359, 1341, 1279, 1240, 1145, 1098, 1060, 957, 947, 842; GPC (DMF+LiBr) Mₙ=1.8 kDa, PDI=1.04. MALDI-TOF MS: molecular ion centered at 2.0 kDa.

MeO-PEG5 kDa-Allyl: 5.00 g (1 mmol) Poly (ethylene glycol) methyl ether (Mₙ=5 kDa) were reacted according to the general procedure (I) and the product was obtained as a white solid (4.45 g), 88% yield. ¹H-NMR (CDCl₃): δ 5.86-5.95 (m, 1H, —CH═CH₂), 5.26 (d, J=17.3 Hz, 1H, trans vinyl —CH═CH₂), 5.17 (d, J=10.3 Hz, 1H, cis vinyl —CH═CH₂), 4.01 (d, J=5.3 Hz, 2H, —O—CH₂—CH═CH₂), 3.44-3.82 (m, 553H, PEG backbone), 3.37 (s, 3H, H₃C—O—); ¹³C-NMR (CDCl₃) δ 134.9, 117.2, 72.3, 72.0, 70.7, 69.5, 59.1; FT-IR, v(cm⁻¹) 2881, 1466, 1360, 1341, 1279, 1240, 1147, 1098, 1060, 959, 842; GPC (DMF+LiBr): Mₙ=5.7 kDa, PDI=1.02.

MeO-PEG10 kDa-Allyl: 2.00 g (0.2 mmol) Poly (ethylene glycol) methyl ether (Mₙ=10 kDa) were reacted according to the general procedure (I) and the product was obtained as a white solid (1.98 g). ¹H-NMR (CDCl₃): δ 5.82-5.95 (m, 1H, —CH═CH₂), 5.25 (dd, J=1.4 Hz, 17.2 Hz, 1H, trans vinyl —CH═CH₂), 5.15 (dd, J=1.1 Hz, 10.3 Hz, 1H, cis vinyl —CH═CH₂), 4.00 (d, J=5.6 Hz, 2H, —O—CH₂—CH═CH₂), 3.43-3.81 (m, 956H, PEG backbone), 3.35 (s, 3H, H₃C—O—); ¹³C-NMR (CDCl₃) δ 134.9, 117.2, 72.3, 72.0, 71.1, 70.7, 69.5, 59.1; FT-IR, v(cm⁻¹): 2881, 1467, 1454, 1360, 1341, 1279, 1240, 1147, 1098, 1060, 960, 948, 842; GPC (DMF+LiBr): Mₙ=11.2 kDa, PDI=1.02.

### General Procedure for Compounds 2a-c

MeO-PEG-Allyl was dissolved in MeOH (5 mL per 1 g). Cystamine hydrochloride (40 eq.) and DMPA (0.2 eq.) were added. The solution was purged with nitrogen for minutes and then placed under UV light at 365 nm for 2 hours. MeOH was evaporated to dryness and the crude mixture was dissolved in NaOH 1N (100 mL per 1 g). This aqueous phase was extracted with DCM (3×50 mL). The organic phase was filtered through celite and evaporated in vacuum. The residue was re-dissolved in DCM (5 mL per 1 g PEG) and product was precipitated by the dropwise addition of 1:1 v/v Ether:Hexane mixture (50 mL per 1 g PEG). The white precipitate was filtered and washed with ether and then with hexane and was dried under high vacuum.

2a: 2.00 g (0.97 mmol) MeO-PEG2k-Allyl were reacted according to the general procedure (II) and the product was obtained as a white solid (1.70 g, 82% yield) ¹H-NMR (CDCl₃): δ 3.44-3.82 (m, 225H, PEG backbone), 3.37 (s, 3H, H₃C—O—), 2.86 (t, J=6.3 Hz, 2H, —CH₂—NH₂), 2.56-2.62 (m, 4H, —CH₂—S—CH₂—), 1.85 (qui, J=6.7 Hz, 2H, —O—CH₂—CH₂—CH₂—S—); ¹³C-NMR (CDCl₃) δ 72.1, 70.7, 70.4, 69.8, 59.2, 41.3, 36.4, 30.0, 28.6; FT-IR v(cm⁻¹): 2883, 1467, 1456, 1360, 1343, 1280, 1241, 1146, 1115, 1061, 963, 947, 842; GPC (DMF+LiBr): =1.8 kDa, PDI=1.04.

2b: 2.12 g (0.42 mmol) MeO-PEGSk-Allyl were reacted according to the general procedure (II) and the product was obtained as a white solid (2.02 g, 94% yield). ¹H-NMR (CDCl₃): δ 3.45-3.83 (m, 590H, PEG backbone), 3.38 (s, 3H, H₃C—O—), 2.87 (t, J=6.2 Hz, 2H, —CH₂—NH₂), 2.57-2.63 (m, 4H, —CH₂—S—CH₂—), 1.82-1.89 (m, 2H, —O—CH₂—CH₂—CH₂—S—); ¹³C-NMR (CDCl₃): δ 72.1, 70.7, 70.3, 69.4, 59.2, 40.6, 36.4, 29.8, 28.5; FT-IR v(cm⁻¹): 2882, 1542, 1466, 1360, 1341, 1279, 1240, 1146, 1102, 1060, 959, 842; GPC (DMF+LiBr): Mₙ=5.6 kDa, PDI=1.04.

2c: 500 mg (0.05 mmol) MeO-PEG10k-Allyl were reacted according to the general procedure (II) and the product was obtained as a white solid (434 mg) 86% yield. ¹H-NMR (CDCl₃): δ 3.43-3.81 (m, 1152H, PEG backbone), 3.36 (s, 3H, H₃C—O—), 2.87 (t, J6.4=Hz, 2H, —CH₂—NH₂), 2.53-2.66 (m, 4H, —CH₂—S—CH₂—), 1.84 (qui, J=6.7 Hz, 2H, —0-CH₂—CH₂—CH₂—S—); ¹³C-NMR (CDCl₃) δ 72.0, 71.2, 70.7, 69.7, 59.1, 41.1, 35.9, 29.9, 28.5; FT-IR v(cm⁻¹): 2880, 1467, 1454, 1359, 1341, 1279, 1240, 1146, 1096, 1060, 960, 947, 841; GPC (DMF+LiBr): Mₙ=11.3 kDa, PDI=1.02.

### General Procedure for Compounds 4a-c

Compounds 2a-c were dissolved in DCM (10 mL per 1 g) followed by addition of compound 3 (3 eq.) and DIPEA (9 eq.) and the reaction was stirred overnight. The solvent was evaporated in vacuum and the crude mixture was loaded on a MeOH based LH20 SEC column. The fractions that contained the product were unified and the MeOH was evaporated in vacuum to yield an oily residue. In order to facilitate the removal of residual MeOH and solidification of the product, the oily residue was re-dissolved in DCM (5 mL per 1 g) followed by addition of hexane (20 mL per 1 g). DCM and hexane were evaporated to dryness and the off-white solid was dried under high vacuum.

4a: 1.27 g (0.98 mmol) 2a was reacted according to the general procedure (III) and the product was obtained as an off-white solid (1.17 g), 84% yield. ¹H-NMR (CDCl₃): δ 7.03 (d, J=2.2 Hz, 2H, arom H), 6.79 (t, J=5.2 Hz, 1H, —NH—CO—), 6.73 (t, J=2.2 Hz, 1H, arom H), 4.71 (d, J=2.3 Hz, 4H, —O—CH₂—CCH), 3.44-3.82 (m, 213H, PEG backbone), 3.37 (s, 3H, H₃C—O—), 2.76 (t, J=6.4 Hz, 2H, —S—CH₂—), 2.64 (t, J=7.2 Hz, 2H, —CH₂—S—), 2.56 (t, J=2.3 Hz, 2H, —CCH), 1.86 (qui, J=6.6 Hz, 2H, —O—CH₂—CH₂—CH₂—S—)¹³C-NMR (CDCl₃) δ 166.9, 158.9, 137.0, 106.8, 105.6, 78.2, 76.2, 72.1, 70.7, 70.3, 69.5, 59.2, 56.3, 39.0, 31.8, 29.8, 28.4; FT-IR v(cm⁻¹): 2882, 1593, 1466, 1455, 1359, 1341, 1279, 1241, 1146, 1103, 1060, 960, 947, 842; GPC (DMF+LiBr): Mₙ=2.0 kDa, PDI=1.03. MALDI-TOF MS: molecular ion centered at 2.3 kDa.

4b: 1.20 g (0.23 mmol) 2b were reacted according to the general procedure (III) and the product was obtained as an off-white solid (1.10 g), 90% yield. ¹H-NMR (CDCl₃): δ 7.04 (d, J=2.1 Hz, 2H, arom H), 6.76-6.81 (m, 1H, —NH—CO—), 6.74 (t, J=2.1 Hz, 1H, arom H), 4.72 (d, J=2.2 Hz, 4H, —O—CH₂—CCH), 3.45-3.83 (m, 567H, PEG backbone), 3.38 (s, 3H, H₃C—O—), 2.77 (t, J=6.4 Hz, 2H, —S—CH₂—), 2.65 (t, J=7.0 Hz, 2H, —CH₂—S—), 2.57 (t, J=2.2 Hz, 2H, —CCH), 1.87 (qui, J=6.7 Hz, 2H, —O—CH₂—CH₂—CH₂—S—); ¹³C-NMR (CDCl₃) δ 166.9, 158.8, 136.9, 106.8, 105.6, 78.2, 76.2, 72.1, 70.7, 70.3, 69.5, 59.1, 56.3, 39.0, 31.8, 29.8, 28.4; FT-IR v(cm⁻¹): 2883, 1654, 1593, 1542, 1467, 1360, 1342, 1279, 1240, 1147, 1107, 1061, 961, 842; GPC (DMF+LiBr): =6.2 kDa, PDI=1.03. MALDI-TOF MS: molecular ion centered at 5.5 kDa.

4c: 200 mg (0.02 mmol) 2c were reacted according to the general procedure (III) and the product was obtained as an off-white solid (202 mg, quantitative yield). ¹H-NMR CDCl₃): δ 7.02 (d, J=2.3 Hz, 2H, arom H), 6.77 (t, J=5.6 Hz, 1H, —NH—CO—), 6.72 (t, J=2.3 Hz, 1H, arom H), 4.69 (d, J=2.4 Hz, 4H, —O—CH₂—CCH), 3.42-3.80 (m, 1089H, PEG backbone), 3.35 (s, 3H, H₃C—O—), 2.74 (t, J=6.5 Hz, 2H, —S—CH₂—), 2.63 (t, J=7.2 Hz, 2H, —CH₂—S—), 2.55 (t, J=2.4 Hz, 2H, —CCH), 1.84 (qui, J=6.7 Hz, 2H, —O—CH₂—CH₂—CH₂—S—); ¹³C-NMR (CDCl₃) δ 166.8, 158.8, 136.9, 106.8, 105.6, 78.1, 76.2, 72.0, 71.1, 70.6, 69.8, 69.4, 59.1, 56.2, 39.0, 31.7, 29.7, 28.3; FT-IR v(cm⁻¹): 2880, 1593, 1359, 1467, 1454, 1341, 1279, 1241, 1146, 1096, 1060, 960, 947, 841; GPC (DMF+LiBr): =11.8 kDa, PDI=1.03.

### General Procedure for Compounds 6a-c

Compounds 4a-c were dissolved in MeOH (5 mL per 1 g). 2-(Boc-amino)-ethanethiol (80 eq.) and DMPA (0.8 eq.) were added. The solution was purged with nitrogen for 15 minutes and then placed under UV light at 365 nm for 2 hours. MeOH was evaporated to a dryness and the crude was loaded on a MeOH based LH20 SEC column. The fractions that contained the product were unified and the MeOH was evaporated in vacuum to yield an oily residue. In order to facilitate the removal of residual MeOH and solidification of the product, the oily residue was re-dissolved in DCM (5 mL per 1 g) followed by addition of Hexane (20 mL per 1 g). DCM and hexane were evaporated to dryness and the off-white solid was dried under high vacuum.

6a: 300 mg (0.13 mmol) 4a were reacted according to the general procedure (IV) and the product was obtained as an off-white solid (357 mg, 91% yield). ¹H-NMR (CDCl₃): δ 6.96-7.04 (m, 3H, arom H+-NH—CO—), 6.58-6.64 (m, 1H, arom H) 5.07-5.25 (m, 4H, —NH-(Boc)), 4.12-4.30 (m, 4H, arom-O—CH₂—), 3.45-3.82 (m, 258H, PEG backbone), 3.37 (s, 3H, H₃C—O—), 3.24-3.35 (m, 8H, —CH₂—NH(Boc)), 3.09-3.19 (m, 2H, —CH—S—), 2.87-2.99 (m, 4H, —CH—CH₂—S—), 2.72-2.87 (m, 6H, —S—CH₂-+-S—CH₂—), 2.59-2.72 (m, 6H, —CH₂—S-+-CH₂—S—), 1.43 (s, 36H, Boc); ¹³C-NMR (CDCl₃) δ 166.9, 159.5, 156.0, 155.9, 136.8, 106.2, 104.9, 79.4, 71.9, 70.6, 70.2, 69.7, 69.5, 59.0, 45.0, 40.5, 40.1, 39.3, 34,4, 33.0, 32.0, 31.5, 29.6, 28.5, 28.3; FT-IR v(cm-1): 2883, 1712, 1592, 1521, 1452, 1391, 1361, 1341, 1279, 1239, 1101, 945, 842; GPC (DMF+LiBr): Mᵣ, =2.5 kDa, PDI=1.04.

6b: 1.01 g (0.19 mmol) 4b were reacted according to the general procedure (IV) and the product was obtained as an off-white solid (1.09 g, 95% yield). ¹H-NMR (CDCl₃): δ 6.92-7.04 (m, 3H, arom H+-NH—CO—), 6.55-6.62 (m, 1H, arom H) 5.15-5.28 (m, 2H, —NH-(Boc)), 5.03-5.15 (m, 2H, —NH-(Boc)), 4.10-4.27 (m, 4H, arom-O—CH₂—), 3.42-3.80 (m, 590H, PEG backbone), 3.21-3.37 (m, 11H, H₃C—O-+-CH₂—NH(Boc)), 3.07-3.19 (m, 2H, —CH—S—), 2.83-2.98 (m, 4H, —CH—CH₂—S—), 2.70-2.83 (m, 6H, —S—CH₂-+-S—CH₂—), 2.55-2.70 (m, 6H, —CH₂—S-+-CH₂—S—), 1.85 (qui, J=6.7 Hz, 2H, —O—CH₂—CH₂—CH₂—S—), 1.41 (s, 36H, Boc); ¹³C-NMR (CDCl₃) δ 167.0, 159.6, 156.0, 155.9, 136.8, 106.2, 105.0, 79.5, 72.0, 71.8, 70.6, 70.3, 69.8, 69.5, 59.1, 45.1, 40.5, 40.2, 39.3, 34.5, 33.1, 32.1, 31.6, 29.7, 28.5, 28.4; FT-IR v(cm-1): 2868, 1706, 1648, 1592, 1522, 1455, 1390, 1364, 1348, 1272, 1250, 1096, 947, 846; GPC (DMF+LiBr): Mₙ=7.2 kDa, PDI=1.03. MALDI-TOF MS: molecular ion centered at 6.0 kDa.

6c: 167 mg (0.02 mmol) 4c were reacted according to the general procedure (IV) and the product was obtained as an off-white solid (170 mg, quantitative yield). ¹H-NMR (CDCl₃): δ 6.92-7.03 (m, 3H, arom H+-NH—CO—), 6.55-6.62 (m, 1H, arom H), 5.04-5.26 (m, 4H, —NH-(Boc)), 4.13-4.27 (m, 4H, arom-O—CH₂—), 3.42-3.80 (m, 1225H, PEG backbone), 3.35 (s, 3H, H₃C—O—), 3.22-3.33 (m, 8H, —CH₂—NH(Boc)), 3.19-3.22 (m, 2H, —CH—S—), 2.85-2.96 (m, 4H, —CH—CH₂—S—), 2.72-2.77 (m, 6H, —S—CH₂-+-S—CH₂—), 2.61-2.70 (m, 6H, —CH₂—S-+-CH₂—S—), 1.85 (qui, J=6.6 Hz, 2H, —O—CH₂—CH₂—CH₂—S—), 1.41 (s, 36H, Boc); ¹³C-NMR (CDC₁₃) δ 167.0, 159.7, 156.03, 155.99, 136.9, 106.3, 105.1, 79.6, 72.1, 70.7, 70.4, 70.3, 69.9, 69.6, 59.1, 45.2, 40.6, 40.2, 39.4, 34.6, 33.1, 32.2, 31.6, 29.8, 28.6, 28.5; FT-IR v(cm⁻¹): 2880, 1710, 1592, 1467, 1454, 1360, 1341, 1279, 1241, 1146, 1097, 1060, 960, 947, 841; GPC (DMF+LiBr): Mₙ=12.8 kDa, PDI=1.02.

### General Procedure for the Amphiphilic Hybrids 1a-c

Compounds 6a-c were dissolved in DCM (5 mL per 1 g) and TFA was added (5 mL per 1 g). After 30 minutes the solution was evaporated to dryness and dried in vacuum. Compounds 7a-c were re-dissolved in DCM (5 mL per 1 g). 4-nitrophenyl 2-phenylacetate, 8, (12 eq.) and DIPEA (36 eq.) was added and the reaction was allowed to stir overnight. The solvent was evaporated to dryness and the crude mixture was loaded on a MeOH based LH20 SEC column. The fractions that contained the product were unified and the MeOH was evaporated in vacuum to yield an oily residue. In order to facilitate the removal of residual MeOH and solidification of the product, the oily residue was re-dissolved in DCM (5 mL per 1 g) followed by addition of hexane (20 mL per 1 g). DCM and hexane were evaporated to dryness and the off-white solid was dried under high vacuum.

1a: 120 mg (0.13 mmol) 6a were reacted according to the general procedure and the product was obtained as an off-white solid (120 mg, quantitative yield). ¹H-NMR (CDCl₃): δ 7.55 (t, J=4.9 Hz, 1H, —NH—CO—), 7.15-7.41 (m, 20H, arom H), 7.00-7.09 (m, 2H, arom H), 6.53-6.61 (m, 1H, arom H), 6.22-6.34 (m, 2H, —NH—CO—CH₂-Ph), (6.08-6.22m, 2H, —NH—CO—CH₂-Ph), 4.03-4.24 (m, 4H, arom-O—CH₂—), 3.54-3.81 (m, 222H, PEG backbone), 3.52 (s, 8H, —NH—CO—CH₂-Ph), 3.27-3.45 (m, 11H, H₃C—O-+-CH₂—NHCO—CH₂-Ph), 2.91-3.15 (m, 2H, —CH—S—), 2.76-2.91 (m, 4H, —CH—CH₂—S—), 2.68-2.76 (m, 6H, —S—CH₂-+-S—CH₂—), 2.51-2.68 (m, 6H, —CH₂—S-+-CH₂—S—), 1.82-1.95 (m, 2H, —O—CH₂—CH₂—CH₂—S—); ¹³C-NMR (CDCl₃) δ 171.6, 171.4, 167.1, 159.5, 137.0, 134.9, 129.5, 129.1, 127.5, 106.5, 105.0, 72.1, 70.7, 70.3, 69.9, 69.7, 59.2, 44.9, 43.8, 39.7, 39.6, 39.2, 34.3, 32.4, 31.6, 31.4, 29.83, 29.79, 28.6; FT-IR v(cm⁻¹): 2884, 1647, 1592, 1466, 1455, 1360, 1341, 1279, 1241, 1146, 1100, 1060, 961, 948, 842; GPC (DMF): Mₙ=2.4 kDa, PDI=1.04; MALDI-TOF MS: molecular ion centered at =3.0 kDa.

1b: 327 mg (0.05 mmol) 6b were reacted according to the general procedure and the product was obtained as an off-white solid (325 mg, quantitative yield). ¹H-NMR (CDCl₃): δ 7.55 (t, J=5.5 Hz, 1H, —NH—CO—), 7.17-7.37 (m, 20H, arom H), 7.04 (d, J=2.1 Hz, 2H, arom H), 6.56 (t, J=2.1 Hz, 1H, arom H), 6.26 (t, J=5.8 Hz, 2H, —NH—CO—CH₂-Ph), 6.17 (t, J=5.7 Hz, 2H, —NH—CO—CH₂-Ph), 4.05-4.20 (m, 4H, arom-O—CH₂—), 3.54-3.82 (m, 570H, PEG backbone), 3.52 (s, 8H, —NH—CO—CH₂-Ph), 3.32-3.43 (m, 11H, H₃C—O-+-CH₂—NHCO—CH₂-Ph), 3.07 (qui, J=6.1 Hz, 2H, —CH—S), 2.76-2.89 (m, 4H, —CH—CH₂—S—), 2.67-2.76 (m, 6H, —S—CH₂-+-S—CH₂—), 2.54-2.67 (m, 6H, —CH₂—S—+-CH₂—S—), 1.85 (qui, J=6.8 Hz, 2H, —O—CH₂—CH₂—CH₂—S—); ¹³C-NMR (CDCl₃) δ 171.5, 171.4, 167.1, 159.5, 137.0, 134.9, 129.5, 129.1, 127.5, 106.5, 104.9, 72.1, 70.7, 70.3, 69.9, 69.7, 59.2, 44.9, 43.8, 39.7, 39.5, 39.2, 34.3, 32.4, 31.4, 29.83, 29.78, 28.6; FT-IR v(cm⁻¹): 2844, 1647,1592, 1466, 1455, 1360, 1341, 1279, 1241, 1146, 1100, 1060, 961, 948, 842; GPC (DMF): Mₙ=7.1 kDa, PDI=1.03; MALDI-TOF MS: molecular ion centered at 6.3 kDa.

1c: 73 mg (0.01 mmol) 6c were reacted according to the general procedure and the product was obtained as an off-white solid (68.5 mg, 93% yield). ¹H-NMR (CDCl₃): δ 7.61 (t, J=5.3 Hz, 1H, —NH—CO-arom), 7.22-7.33 (m, 20H, arom H), 7.02-7.08 (m, 2H, arom H), 6.55-6.61 (m, 1H, arom H), 6.41 (t, J=5.4 Hz, 2H, —NH—CO—CH₂-Ph), 6.31 (t, J=5.1 Hz, 2H, —NH—CO—CH₂-Ph), 4.08-4.19 (m, 4H, arom-O—CH₂—), 3.53-3.83 (m, 1212H, PEG backbone), 3.52 (s, 8H, —NHCO—CH₂-Ph), 3.36-3.42 (m, 11H, H₃C—O-+-CH₂—NHCO—CH₂-Ph), 3.09 (qui, J=5.7 Hz, 2H, —CH—S—), 2.77-2.87 (m, 4H, —CH—CH₂—S), 2.67-2.77 (m, 6H, —S—CH₂-+-S—CH₂—), 2.58-2.67 (m, 6H, —CH₂—S-+-CH₂—S—), 1.86 (qui, J=6.6 Hz, 2H, —O—CH₂—CH₂—CH₂—S—); ¹³C-NMR (CDCl₃) δ 171.4, 171.3, 167.0, 159.4, 136.9, 134.9, 129.4, 128.9, 127.3, 106.4, 104.8, 82.1, 74.0, 72.9, 71.9, 71.7, 70.6, 70.2, 69.7, 69.6, 69.4, 68.3, 67.1, 64.8, 63.7, 59.0, 44.7, 43.6, 39.6, 39.5, 39.1, 34.2, 32.2, 31.4, 31.2, 9.68, 29.65, 28.5; FT-IR v(cm-1): 2884, 1658, 1591, 1467, 1454, 1359, 1341, 1279, 1240, 1146, 1098, 1060, 960, 947, 841; GPC (DMF+LiBr): Mₙ=12.7 kDa, PDI=1.02.

### Synthesis of 4-nitrophenyl 3,5-bis(prop-2-yn-1-yloxy) benzoate (3)

3,5-bis(prop-2-yn-1-yloxy) benzoic acid (1.70 gr, 7.4 mmol) and 4-nitrophenol (1.13 g, 8.12 mmol, 1.1 eq) were dissolved in EtOAc (20 ml). Flask was cooled to 0° C. and DCC (1.68 g, 8.12 mmol, 1.1 eq) was added. After 3 hours the solution was filtered off and EtOAc was removed in vacuum. Crude mixture was purified by a silica column using 100% DCM as an eluent and the product was obtained as white solid in 71% yield (1.84 gr)¹H-NMR (CDCl₃): δ 8.33 (d, J=9.0 Hz, 2H, arom H), 7.45 (d, J=2.2 Hz, 2H, arom H), 7.41 (d, J=9.0 Hz, 2H, arom H), 6.93 (t, J=2.2 Hz, 1H, arom H), 4.76 (d, J=2.2 Hz, 4H, —O—CH₂—CCH), 2.57 (t, J=2.2 Hz, —CCH)¹³C-NMR (CDCl₃): δ 163.8, 159.0, 155.8, 145.7, 130.7, 125.5, 122.7, 109.9, 108.7, 77.9, 76.4, 56.4; FT-IR, v(cm⁻¹) 3270, 2117, 1747, 1610, 1590, 1522, 1506, 1491, 1469, 1451, 1387, 1357, 1335, 1294, 1271, 1216, 1202, 1161, 1115, 1093, 1068, 1029, 992, 948, 937, 910, 895, 859, 844, 814; HR-MS (ESI) calculated for C₁₉H₁₄NO₆ 352.0816 (MH⁺), found 352.0817.

### Synthesis of 4-nitrophenyl-2-phenylacetate (8)

Phenyl acetic acid (5.00 g, 36.7 mmol) and 4-nitrophenol (5.60 g, 40.4 mmol, 1.1 eq) were dissolved in EtOAc (50 ml). Flask was cooled to 0° C. and DCC (8.30)g, 40.4 mmol, 1.1 eq) was added. After 3 hours the solution was filtered off and EtOAc was removed in vacuum. Crude mixture was purified by a silica column using 100% DCM as an eluent and the product was obtained as white solid in 77% yield (7.78 g). ¹H-NMR (CDCl₃): δ 8.25 (d, J=9 Hz, 2H, arom H), 7.32-7.45 (m, 5H, arom H), 7.26 (d, J=9 Hz, 2H, arom H), 3.90 (s, 2H, —CO—CH₂-Ph); ¹³C-NMR (CDCl₃) M69.2, 155.6, 145.5, 132.8, 129.4, 129.0, 127.8, 125.3, 122.5, 41.5; FT-IR, v(cm⁻¹) 1754, 1612, 1589, 1516, 1500, 1486, 1455, 1408, 1351, 1339, 1216, 1203, 1160, 1106, 1078, 1011, 1002, 937, 915, 870, 854.

### Example 3

### Synthesis Protocol of the Amphiphilic PEG-Dendron Hybrids (11b and 15b)

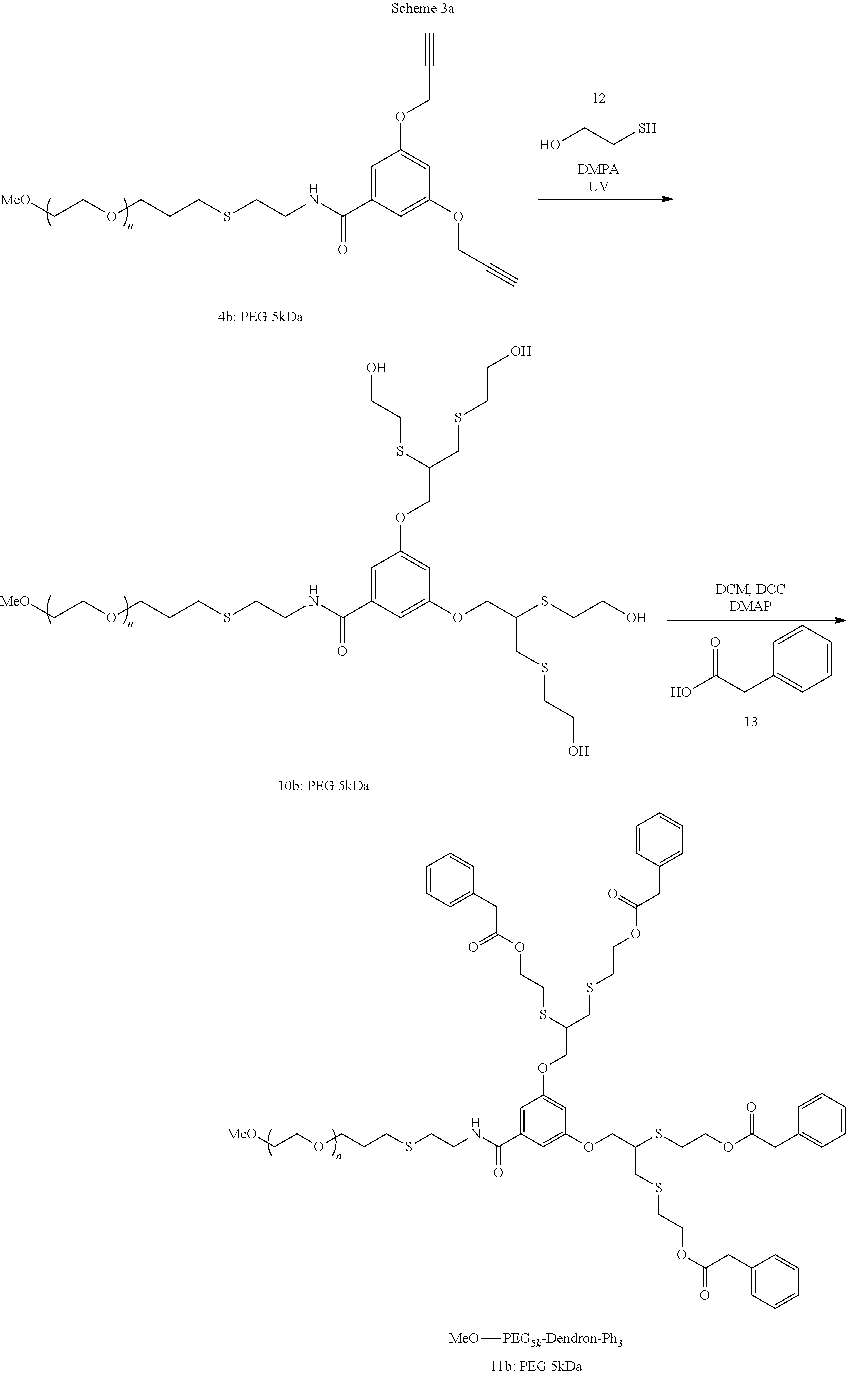

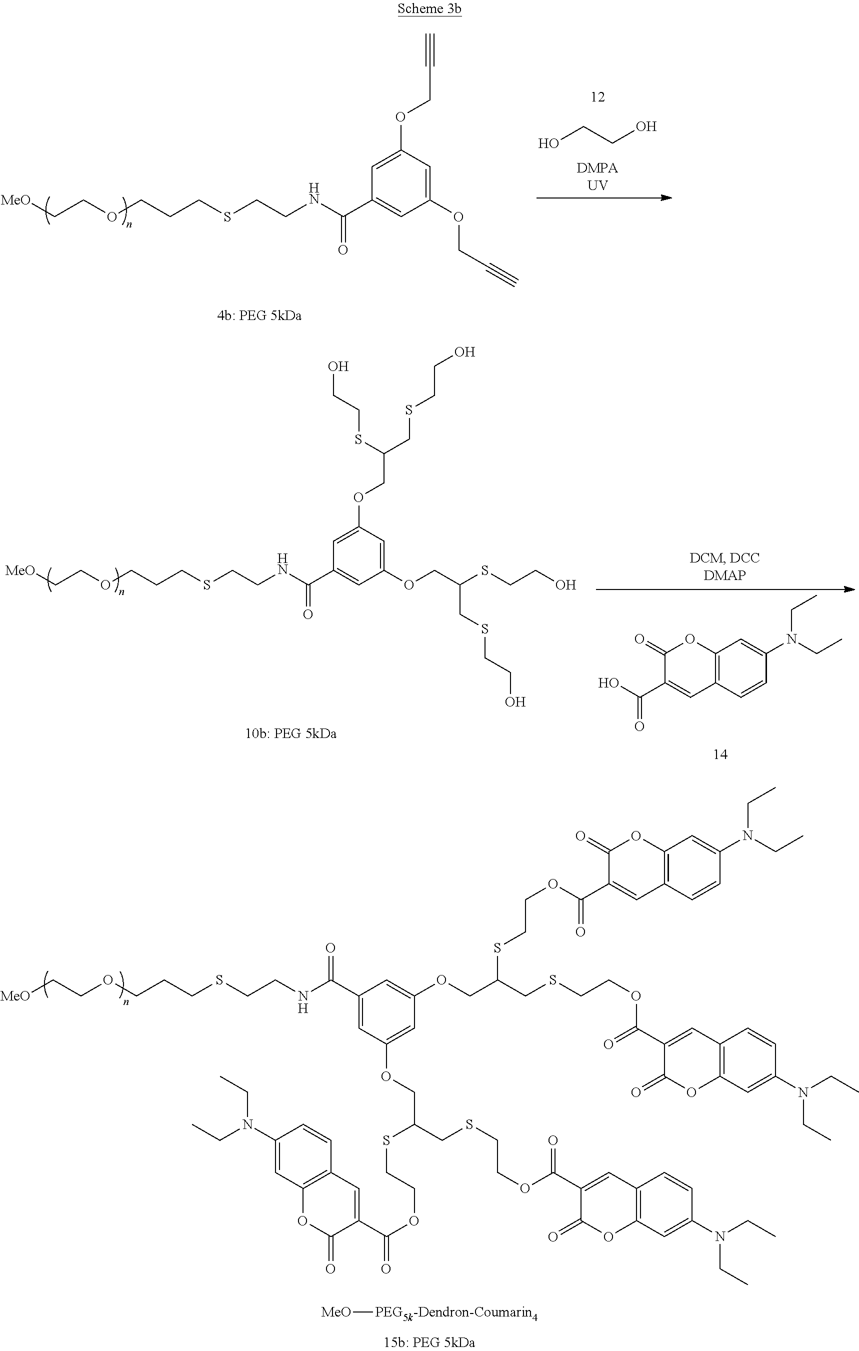

The compounds (11b and 15b) of the invention may be prepared by the process described in general Schemes 3a and 3b hereinabove. Briefly, the hybrid block copolymers were synthesized utilizing mono-methyl ether PEG-amine, 2b, prepared as described in Example 2. Conjugation of compound 2b with an active ester of 3,5-bis(prop-2-yn-1-yloxy)benzoic acid yielded PEG-di-yne, 4b. The latter was further modified by thiol-yne reaction with 2-mercaptoethanol, 12, to give tetra-functionalized PEG-dendron, 10b. In the last step of the synthesis, phenyl acetic acid, 13, or coumarin, 14, were used to introduce the enzyme cleavable hydrophobic surface-groups and to obtain the PEG-dendron hybrids, 11b and 15b respectively.

Similar reactions are performed with 2 kDa PEG and 10 kDa PEG to yield the corresponding PEG-dendron hybrids 11a, 11c (hybrids with phenyl acetic acid), and 15a and 15c (hybrids with coumarin), respectively.

10b: PEG-di acetylene derivative 4b (418 mg, 78.42 μmol) was dissolved in MeOH (2.5 ml). 2-Mercaptoethanol, 12 (80 eq.) and DMPA (0.8 eq.) were added. The solution was purged with nitrogen for 15 minutes and then placed under UV light at 365 nm for 2 hours. MeOH was evaporated to a dryness and the crude was loaded on a MeOH based LH20 SEC column. The fractions that contained the product were unified and the MeOH was evaporated in vacuum to yield an oily residue. In order to facilitate the removal of residual MeOH and solidification of the product, the oily residue was re-dissolved in DCM (5 mL per 1 g) followed by addition of hexane (20 mL per 1 g). DCM and hexane were evaporated to dryness and the off-white solid was dried under high vacuum (386 mg, 87% yield). ¹H-NMR (CDCl₃): δ 7.1 (m, 1H—NH—CO—), 7.00 (m, 2H, arom H) 6.62 (m, 1H, arom H), 4.17-4.28 (m, 4H, arom-O—CH₂—), 3.43-3.89 (m, 535H, PEG backbone+H₂C—OH), 3.36 (s, 3H, H₃C—O—), 3.26-3.29 (m, 2H, —CH—S—), 2.73-3.01 (m, 14H, —CH—CH₂—S-+-S—CH₂—), 2.64 (t, J=7.2 Hz, 2H, —CH₂—S—) 1.85 (qui, J=6.7 Hz, 2H, —O—CH₂—CH₂—CH₂—S—); ¹³C-NMR (CDCl₃) δ 167.2, 159.6, 136.9, 106.3, 105.4, 72.0, 70.7, 70.3, 70.2, 69.5, 61.8, 61.2, 60.5, 59.1, 45.4, 41.4, 39.3, 36.3, 35.3, 35.0, 31.7, 29.8, 29.7, 28.5; FT-IR v(cm⁻¹): 2889, 1592, 1541, 1468, 1360, 1341, 1279, 1240, 1105, 945, 842; GPC (DMF+LiBr): Mₙ=7.1 kDa, PDI=1.03. MALDI-TOF MS: molecular ion centered at 5.7 kDa. Expected Mn=5.6KDa.

11b: 10b (99 mg, 17.7 μmol) was dissolved in dry DCM (3 mL), phenyl acetic acid, 13 (12 eq) was added. The solution was cooled to 0° C. DCC (12 eq) and DMAP (catalytic) were dissolved in dry DCM (100 μL per 20 mg, cooled to 0° C. and then added to the reaction. The reaction was heated to 30° C. and allowed to stir overnight. The crude was filtered and evaporated to dryness. The crude mixture was loaded on a MeOH based LH20 SEC column. The fractions that contained the product were unified and the MeOH was evaporated in vacuum to yield an oily residue. In order to facilitate the removal of residual MeOH and solidification of the product, the oily residue was re-dissolved in DCM (5 mL per 1 g) followed by addition of hexane (20 mL per 1 g). DCM and hexane were evaporated to dryness and the off-white solid was dried under high vacuum (68 mg, 63% yield). ¹H-NMR (CDCl₃): δ 7.22-7.30 (m, 20H, arom H), 6.95 (d, J=1.9 Hz, 2H, arom H), 6.86 (t, J=5.6 Hz 1H—NH—CO—), 6.56 (t, 1H, arom H), 4.21-4.27 (m, 8H, CH₂—O—CO—CH₂-Ph), 4.06-4.16 (m, 4H, arom-O—CH₂—), 3.43-3.81 (m, 512H, PEG backbone), 3.36 (s, 3H, H₃C—O—), 3.1-3.13 (m, 2H, —CH—S—), 2.65-2.94 (m, 14H, —CH—CH₂—S-+-S—CH₂—), 2.61 (t, J=7.2 Hz, 2H, —CH₂—S—) 1.84 (qui, J=6.8 Hz, 2H, —O—CH₂—CH₂—CH₂—S—); ¹³C-NMR (CDCl₃) δ 171.6, 171.5, 166.9, 159.6, 136.9, 133.82, 133.79, 129.4, 128.7 127.3, 127.28, 106.3, 104.6, 72.0, 70.7, 70.3, 70.2, 69.8, 69.6, 64.2, 63.9, 59.1, 45.46, 41.32, 41.31, 39.3, 36.3, 34.9, 31.5, 30.4, 29.8, 29.7, 28.4; FT-IR v(cm⁻¹): 2890,1735, 1592, 1537, 1468, 1360, 1341, 1279, 1239, 1107, 945, 842; GPC (DMF+LiBr): Mₙ=7.1 kDa, PDI=1.03. MALDI-TOF MS: molecular ion centered at 6.2 kDa. Expected Mn=6.1KDa.

15b: compound 10b (100 mg, 17.7 μmol) was dissolved in dry DCM (3 mL), Coumarin, 14 (40 eq) was added. The solution was cooled to 0° C. DCC (40 eq) and DMAP (catalytic) were dissolved in dry DCM (100 μL per 20 mg, cooled to 0° C. and then added to the reaction. The reaction was heated to 30° C. and allowed to stir overnight. The crude was filtered and evaporated to dryness. The crude mixture was loaded on a MeOH:DCM (1:1) based LH20 SEC column. The fractions that contained the product were unified and the solvent was evaporated in vacuum to yield an oily residue. In order to facilitate the removal of residual MeOH and solidification of the product, the oily residue was re-dissolved in DCM (5 mL per 1 g) followed by addition of hexane (20 mL per 1 g). DCM and hexane were evaporated to dryness and the off-white solid was dried under high vacuum (71 mg, 61% yield). ¹H-NMR (CDCl₃): δ 8.39 (s, 2H, arom H), 8.37 (s, 2H, arom H), 7.4 (m, 1H—NH—CO—), 7.32 (d, J=2 Hz 2H, arom H), 7.30 (d, J=2 Hz 2H, arom H), 7.01 (d, J=1.9 Hz, 2H, arom H), 6.62 (t, 1H, arom H), 6.57 (dd J=1.1 Hz 2H, arom H), 6.55 (dd J=1.1 Hz 2H, arom H), 6.38 (s, 2H, arom H), 6.37 (s, 2H, arom H), 4.41-4.48 (m, 8H, CH₂—O—CO—CH₂-Ph), 4.17-4.28 (m, 4H, arom-O—CH₂—), 3.49-3.80 (m, 632H, PEG backbone), 3.38-3.45 (q, 16H, N—CH₂—CH₃—), 3.35 (s, 3H, H₃C—O—), 2.75-3.1 (m, 16H, —CH—S-+-CH—CH₂—S-+-S—CH₂—), 2.63 (t, J=7.2 Hz, 2H, —CH₂—S—) 1.84 (qui, J=7.1 Hz, 2H, —O—CH₂—CH₂—CH₂—S—), 1.18-1.21 (t, 24H, N—CH₂—CH₃—); ¹³C-NMR (CDCl₃) δ 167.1, 163.9, 163.8, 159.6, 158.57, 158.56, 158.28, 158.25, 153.14, 153.12, 149.67, 149.56, 136.8, 131.4, 131.38, 109.75, 109.74, 108.13, 108.06, 107.77, 107.75 106.5, 104.4, 99.5, 96.7, 72.0, 70.6, 70.3, 70.0, 69.7, 64.5, 64.1, 59.1, 45.55, 45.18, 39.8, 35.0, 31.5, 31.3, 30.4, 29.8, 29.7, 28.4, 12.5; FT-IR v(cm⁻¹): 2890, 1756, 1583, 1512, 1468, 1359, 1341, 1279, 1241, 1101, 945, 842; GPC (DMF+LiBr): Mₙ=7.3 kDa, PDI=1.03. MALDI-TOF MS: molecular ion centered at 6.7 kDa. Expected Mn=6.6KDa.

### Example 4

Critical Micelle Concentration (CMC) Measurements of the Amphiphilic PEG-Dendron Hybrids (1a-c)

The Hybrids of the invention were evaluated in their ability to self-assemble into micelles. This was examined by utilizing solubilization experiments with the solvatochromic hydrophobic dye Nile red. All the CMC measurements were performed according to the general procedure described in Example 1. The amphiphilic PEG-dendron hybrids 1a-c were found to self-assemble into micelle with critical micelle concentration of 7.2 μM, 12.4 μM, 21.7 μM, respectively (see also, Table 1; Example 5).

### Example 5

Dynamic Light Scattering (DLS) Measurements of the Amphiphilic PEG-Dendron Hybrids (1a-c)

The self-assembly and disassembly of the PEG-dendron hybrids 1a-c were studied using dynamic light scattering (DLS).

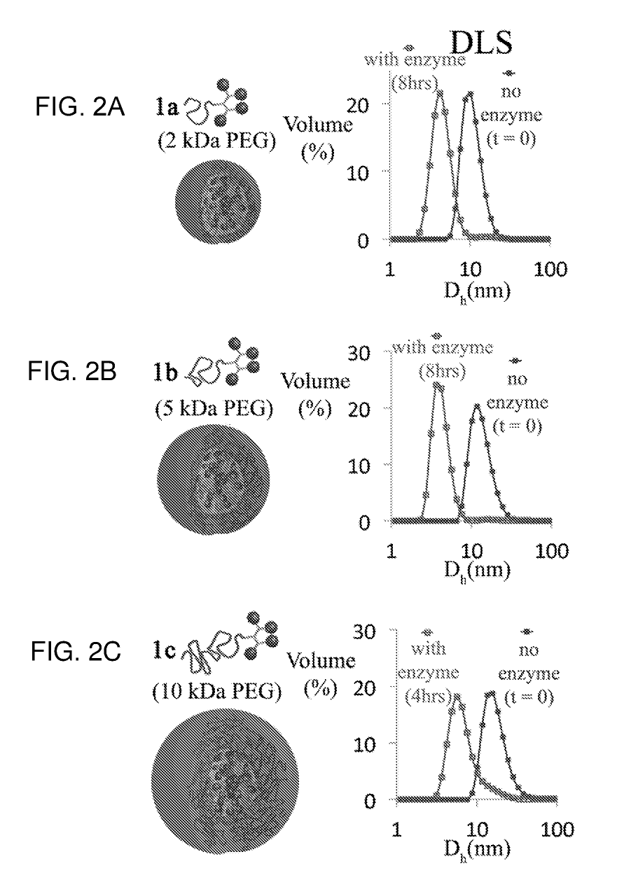

As shown in FIG. 2A-2C (right; 1a-c; t=0, no enzyme) and Table 1, at a concentration of 160 μM the hybrid self-assembled into micelles having a diameters of 11 nm, 14 nm and 18 nm for hybrids 1a-c, respectively.

TABLE 1
CMCs and micelles diameters for PEG-dendron hybrids 1a-c.

| | | | |
|---|---|---|---|
| Hybrid | 1a | 1b | 1c |

| | | | | | | |
|---|---|---|---|---|---|---|
| CMC^{a} | 7.2 | μM | 12.4 | μM | 21.7 | μM |
| Micelle diameter^{b} | 11 | nm | 14 | nm | 18 | nm |

^{a}Calculated by using Nile red.
^{b}Diameters of the micelles as measured by DLS.

Further support for the formation of micelles with a PEG-shell and dendron-based core was obtained from ¹H NMR spectra of PEG-dendron hybrids in D₂O, which showed only the peaks of the PEG's protons (data not shown).

The dissociation of the three micelles of 1a-c was also examined in response to enzymatic activity using DLS, fluorescence spectroscopy, and HPLC. The enzymatic cleavage of the hydrophobic ligand phenyl acetamide should decrease the hydrophobicity of the dendron and destabilize the micelles, leading to their disassembly into the corresponding monomeric hybrids. Indeed, as show in FIG. 2A-2C (right; 1a-c; t=8 hr, with enzyme) the DLS measurements of solutions of the amphiphilic hybrids in the presence of PGA clearly indicated a decrease of the peak that correlates with the larger micellar aggregates and the formation of a new peak, which correlates to the smaller sizes of the non-assembled monomeric chains and the enzyme.

In addition, the PGA enzyme specificity was verified using the micelle based on the amphiphilic Boc protected hybrid 6b as control. The micelle was found stable as no disassembly or degradation was observed (data not shown).

### Example 6

Transmission Electron Microscopy (TEM) Measurements of the Micelles Comprising the Amphiphilic PEG-Dendron Hybrid (1a-c)

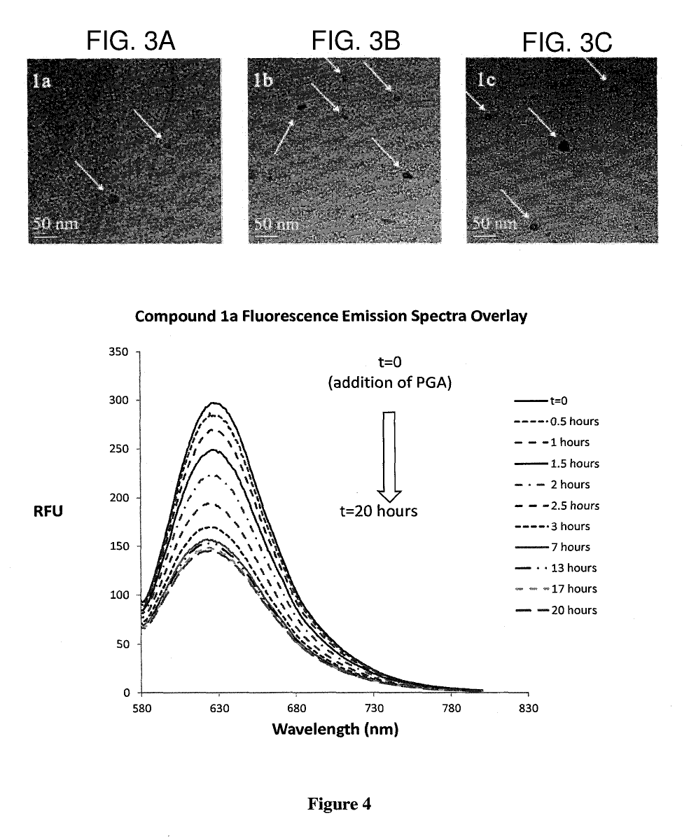

As shown in FIG. 3A-3C, using Transmission Electron Microscopy, the self-assembly of the three PEG-dendron hybrids 1a-c into spherical micelles was confirmed.

### Example 7

Monitoring Micelles (1a-c) Disassembly Using Nile Red Fluorescence

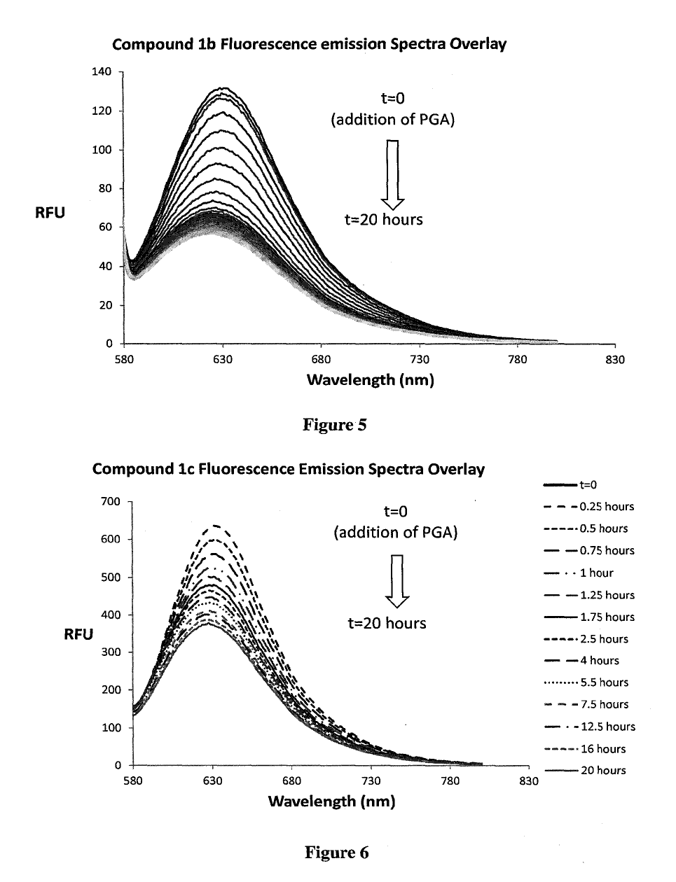

The enzyme responsive disassembly was further supported by change in the fluorescence of encapsulated Nile red dyes. As the dye molecules are released into the aqueous environment upon the disassembly of the micelles, their fluorescence intensity is expected to decrease. As anticipated, time dependent decrease in fluorescence was observed for all three PEG-dendron hybrids, 1a-c, indicating that the Nile red molecules are released from the hydrophobic cores of the micelles as they disassemble upon the addition of the activating enzyme (FIGS. 4-6).

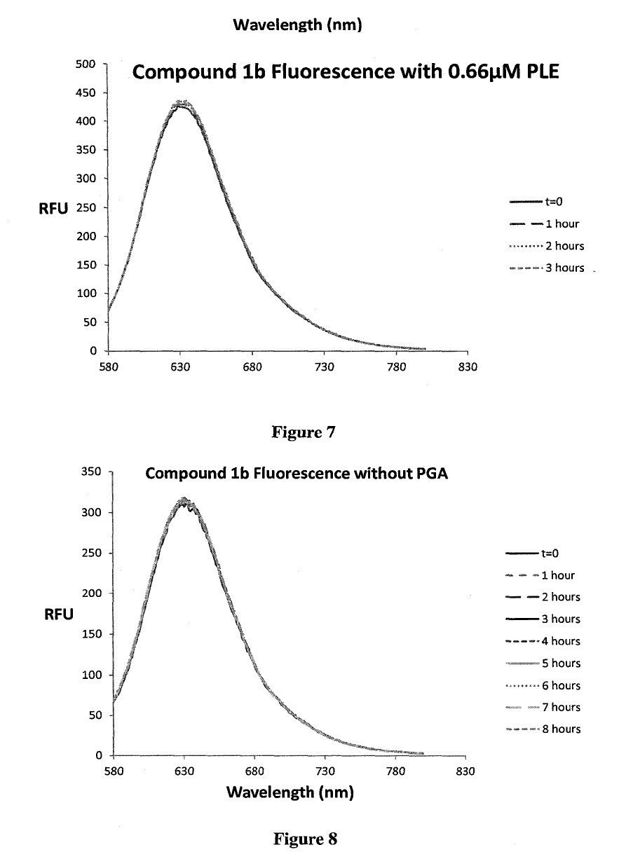

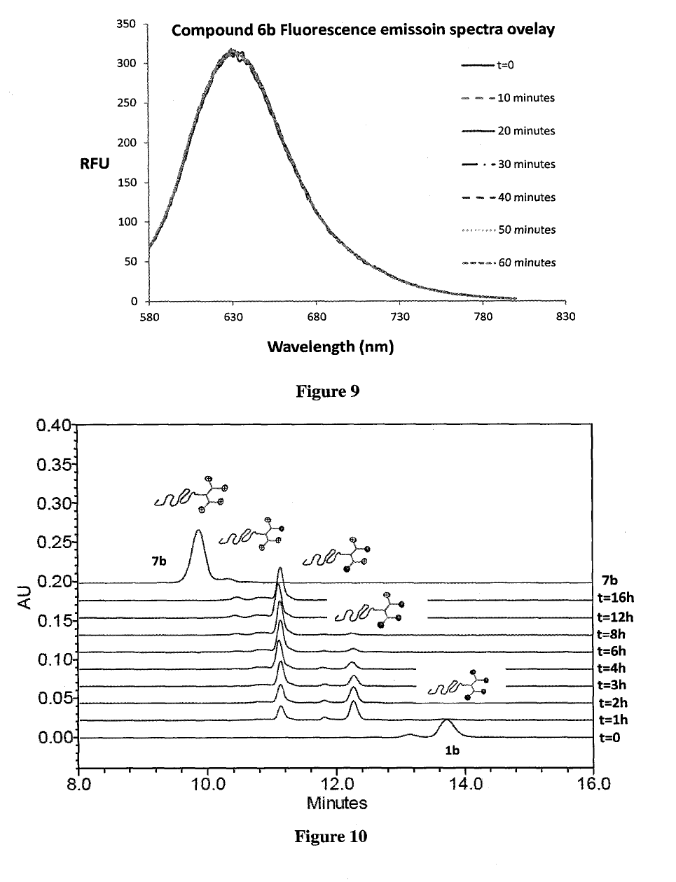

Three control experiments were also performed. The first confirmed the linker by incubating compound 1b with 0.66 μM OF PLE. As this enzyme is not capable of breaking amide bond, the fluorescence emission intensity of compound 1b was not affected (FIG. 7). In the second assay, 1b was incubated in the buffer assay for 12 hours in the absence of the activating enzyme PGA. The fluorescence emission intensity of 1b did not change as no enzymatic cleavage occurred and the micelle remained intact (FIG. 8). The final control experiment involved incubation of the synthetic intermediate, 6b, that has as surface groups Boc protected amine (i.e., carbamate linkage) with the activating enzyme PGA for 1 hour. The fluorescence emission intensity of compound 6b did not change as this enzyme is not able to cleave carbamate linkage (FIG. 9).

### Example 8

Enzymatic Degradation of the Micelles Comprising the Amphiphilic PEG-Dendron Hybrid (1a-c) Monitored by HPLC

The general procedure as depict in Example 1 was employed with the following supplementations:

Enzymatic cleavage of 1b in the presence of 1.4 μM PGA enzyme: 1505 μL of 1b and 15 μL of PGA stock solution reacted according to the general procedure in example 1. Enzymatic degradation was monitored at t=0, 0.5h, 1h, 2h, 4h, 8h, 12h, 28h, 40h and 90h. Compound 7b injection is shown last for comparison.
For enzymatic cleavage of 1b in the presence of 0.14 μM PGA enzyme: 1520 μL of 1b and 1.5 μL of PGA reacted according to the general procedure in example 1. Enzymatic degradation was monitored at t=0, 1h, 2h, 3h, 4h, 6h, 8h, 12h and 16h. Compound 7b injection is shown last for comparison.

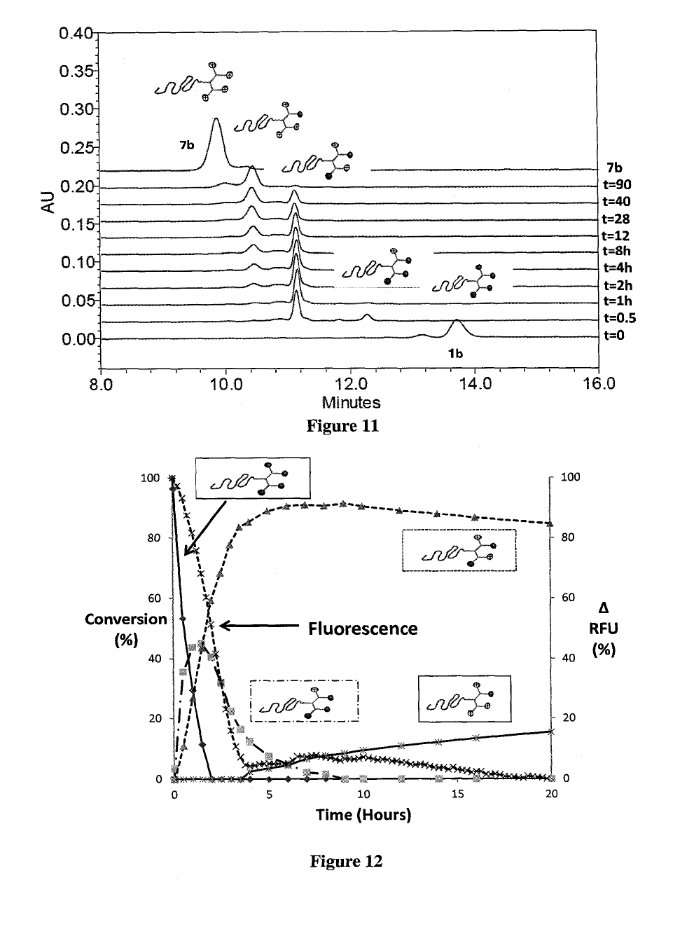

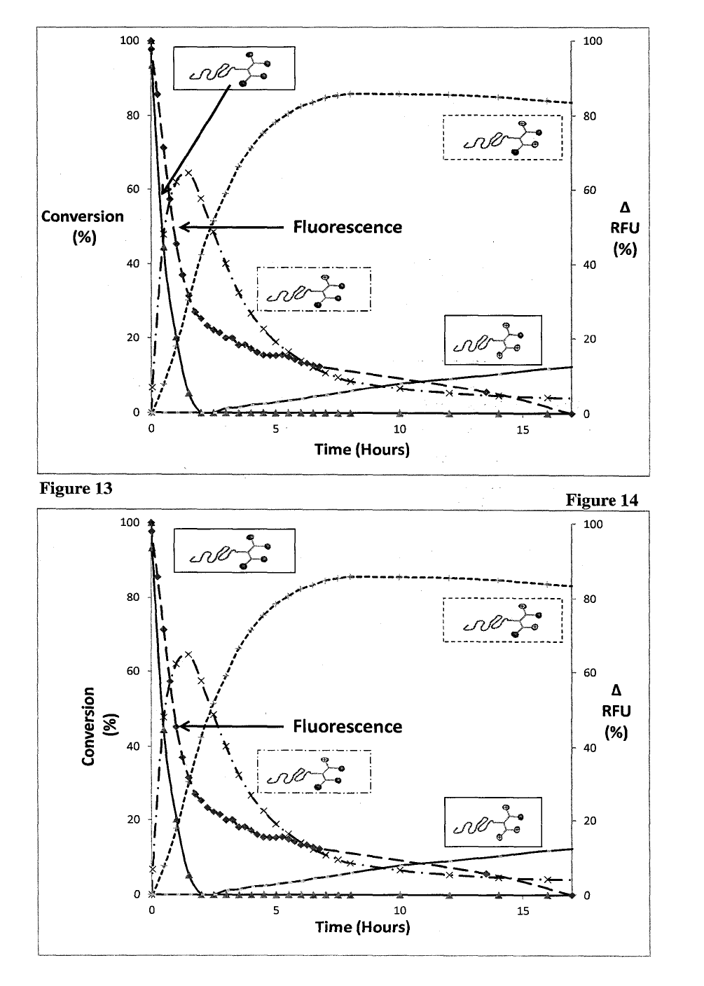

The HPLC analysis revealed a relatively fast disappearance of the amphiphilic hybrids, 1a-c upon incubation with the activating enzyme. Furthermore, only three major intermediates of increasing polarity were formed (FIGS. 10-11). Based on their relative polarities, rate of formation, monodispersity and symmetry of the dendron, these intermediates are most likely partially cleaved hybrids with three, two and one phenyl acetamide end-groups. Comparisons of the HPLC and fluorescence data show good correlations between the decrease in fluorescence and the disappearance of the tetra-functionalized hybrid (i.e. hybrids 1a-c) and the first intermediate with three hydrophobic end-groups (FIGS. 12-14). These correlations indicate that only these two molecular species contribute to the formation of the micelles. Once their concentrations decrease, the micelles disassemble and their fluorescent cargo is released. The formation of the fully degraded tetra-amine hybrids, 7a-c, was not observed even after 24 hours at this relatively low enzyme-concentration (0.14 μM). However, when higher enzyme concentration was applied (1.4 μM), the formation of fully degraded hybrids was observed (FIG. 11).

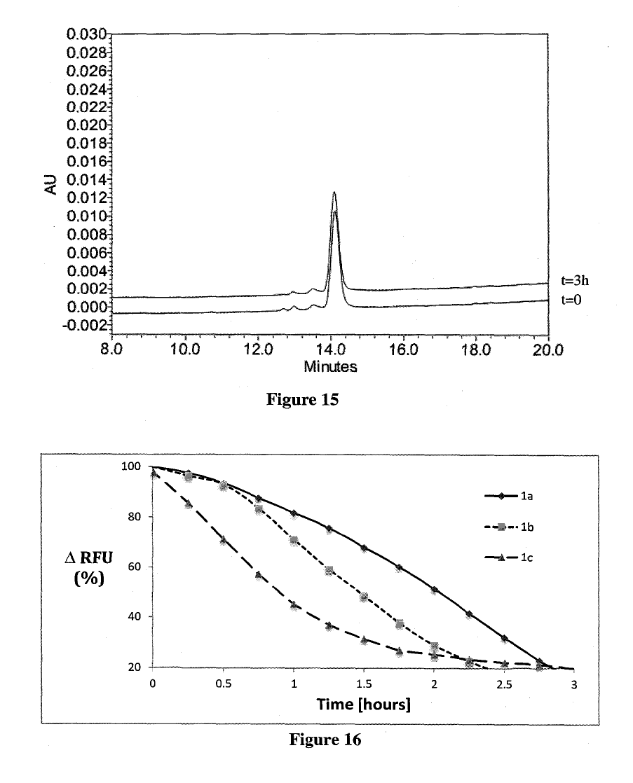

As control experiment, micelles based on hybrid 1b were incubated with an esterase that cannot break amide bonds (PLE), in order to examine the selectivity of the enzymatic activation. The micelle was found to be stable as no disassembly or degradation was observed by HPLC (FIG. 15).

Micelles based on amphiphilic Boc protected hybrids 6a-c were utilized as control experiments and were found by HPLC to be completely stable in the presence of the activating enzyme (data not shown). This further supports the presence of a PEG shell that gives the micelles stealth properties and helps to avoid non-specific activation due to binding to proteins.

### Example 9

### Hydrolysis Rate and Molecular Mechanism of the Disassembly of the Micelles 1a-c

As shown in FIG. 16, the hybrid 1c with the longest PEG chain exhibited a faster cleavage and disassembly in comparison with the micelles with thinner PEG shells (shorter PEG chain, i.e., 1a).

### Example 10

Critical Micelle Concentration (CMC) Measurements of the amphiphilic PEG-dendron hybrids (11b and 15b)

The Critical Micelle Concentration (CMC) measurement was performed on 11b and 15b as depicted in Example 1. The amphiphilic PEG-dendron hybrids 11b and 15b exhibited self-assembly into micelle with critical micelle concentrations of 2.7 μM and 3.6 μM, respectively.

### Example 11

### Dynamic Light Scattering (DLS) Measurement of the Amphiphilic PEG-Dendron Hybrid (11b)

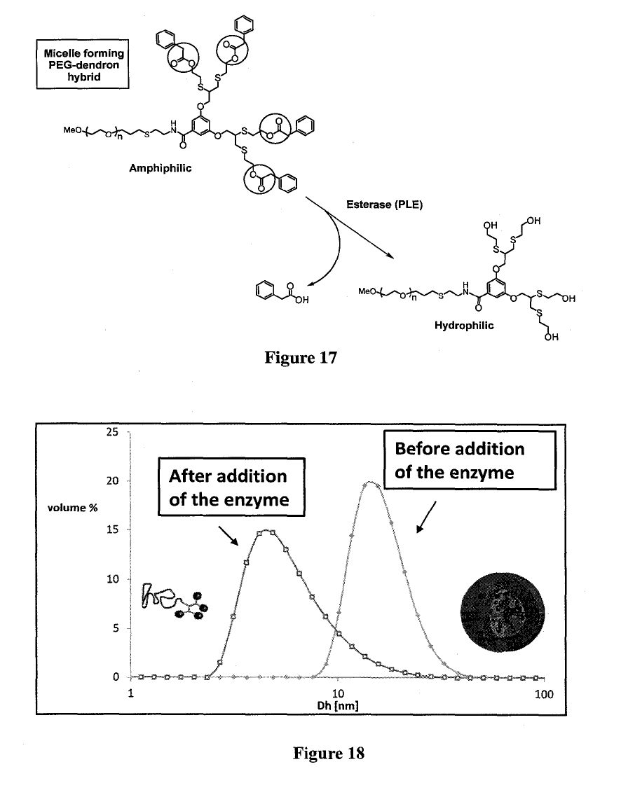

The disassembly of the PEG-dendron 11b in response to enzymatic stimuli (FIG. 17) was studied using DLS according to the procedure depicted in Example 1. As illustrated in FIG. 18, upon the addition of the PLE enzyme, a similar result was seen, which conform to the findings obtained in example 6. The particle size of 11b according to DLS] is ˜17 nm.

### Example 12

¹H-NMR Measurement of the Micelle based on the Amphiphilic PEG-Dendron Hybrid (11b)

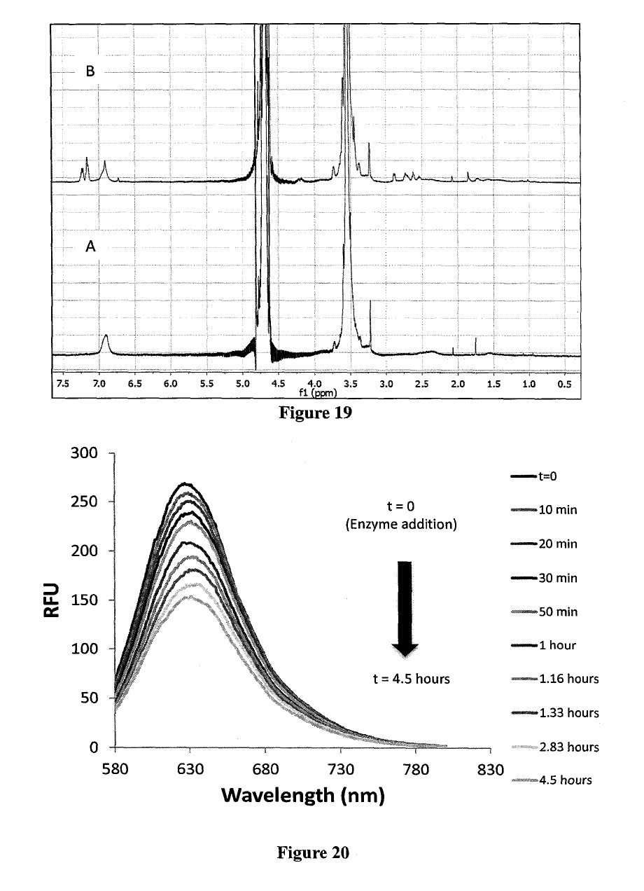

The formation of micelles with a PEG-shell and dendron-based core was verified using ¹H-NMR. The measurement of the PEG-dendron hybrids (11b) in D₂O, showed mostly the peak of the PEG's protons, as expected (FIG. 19; A). Following the addition of the activating enzyme, ¹H-NMR was measured again, and showed the reappearance of the dendron's proton, indicating that the dendron became hydrophilic due to the cleavage of its hydrophobic end-groups, thus further demonstrating the disassembly of the nanocarrier (FIG. 19; B).

### Example 13

Monitoring Micelle (11b and 15b) Disassembly with Nile Red Fluorescence

The disassembly of micelles based on 11b and 15b hybrids comprising the ester cleavable moiety was examined using the encapsulated Nile red dye. The protocol in Example 1 was implemented.

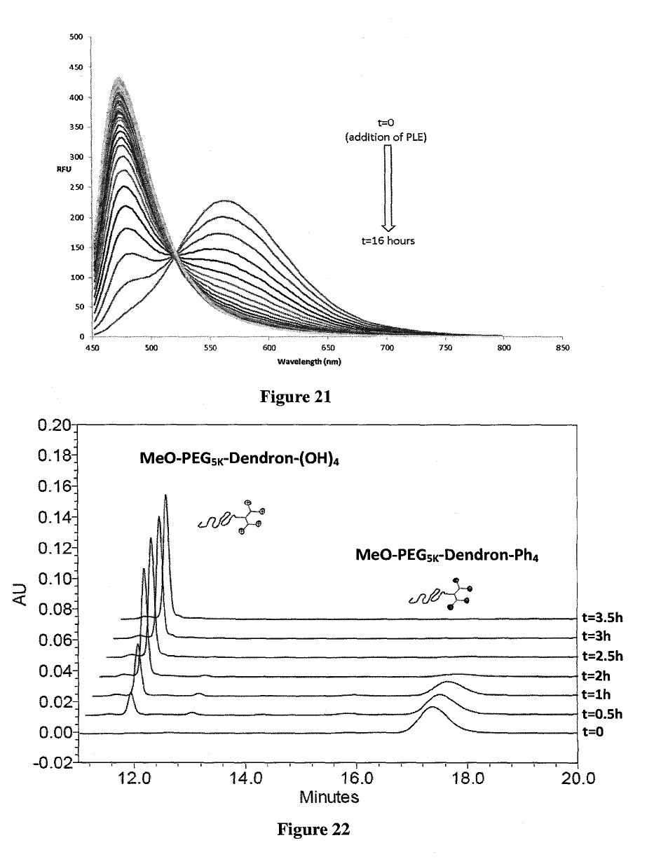

As anticipated, time dependent decrease in fluorescence was observed for PEG-dendron hybrids, 11b and 15b, indicating that the Nile red molecules are released from the hydrophobic cores of the micelles as they disassemble upon the addition of the activating enzyme (FIGS. 20-21).

### Example 14

### Enzymatic Degradation of the Micelles Comprising the Amphiphilic PEG-Dendron Hybrid (10b and 11b) Monitored by HPLC

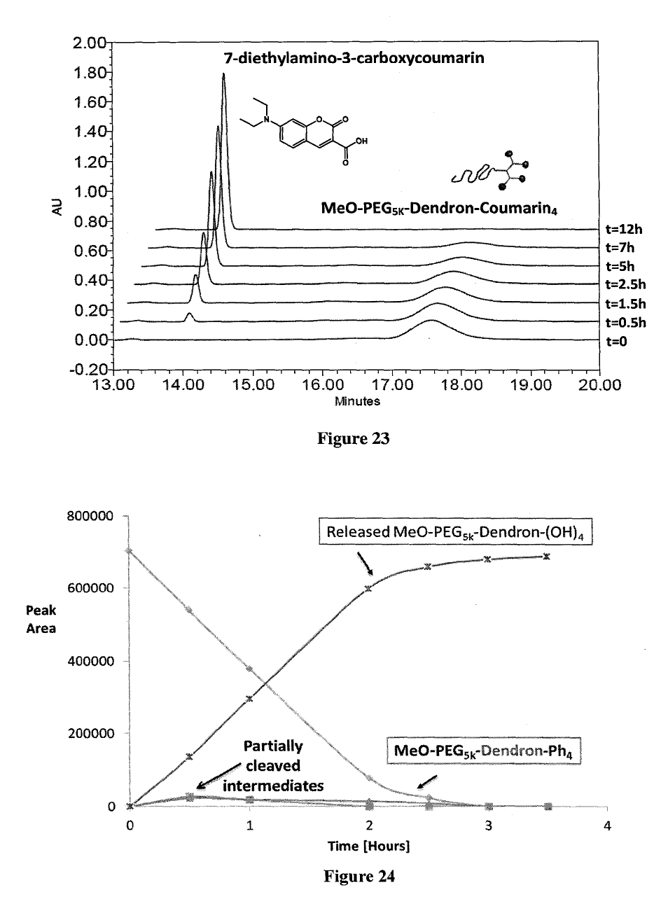

As shown in FIGS. 22-25, HPLC analysis revealed the full disappearance of the amphiphilic hybrids, 11b and 15b upon incubation with the activating enzyme PLE. In the case of hybrid 11b (160 μM), the formation of the fully degraded tetra-hydroxy hybrid, 10b, was observed after less than 3 hours at an enzyme-concentration of 0.23 μM. In the case of hybrid 15b (40 μM), full degradation was observed after nearly 12 hours and required higher concentration of the enzyme PLE (8.5 μM). Notably, for both esterase responsive hybrids 11b and 15b, almost no accumulations of partially cleaved intermediates were observed by the HPLC analysis.

FIG. 26, show the overlay of the HPLC data and the decrease in Nile red fluorescence, which is indicative of the disassembly of the micelles. As can be clearly seen from the obtained results, there is a great correlation between the enzymatic degradation rate of the amphiphilic hybrid 11b and the disassembly rate of the micelles, as indicated by the release of the encapsulated Nile red dyes.

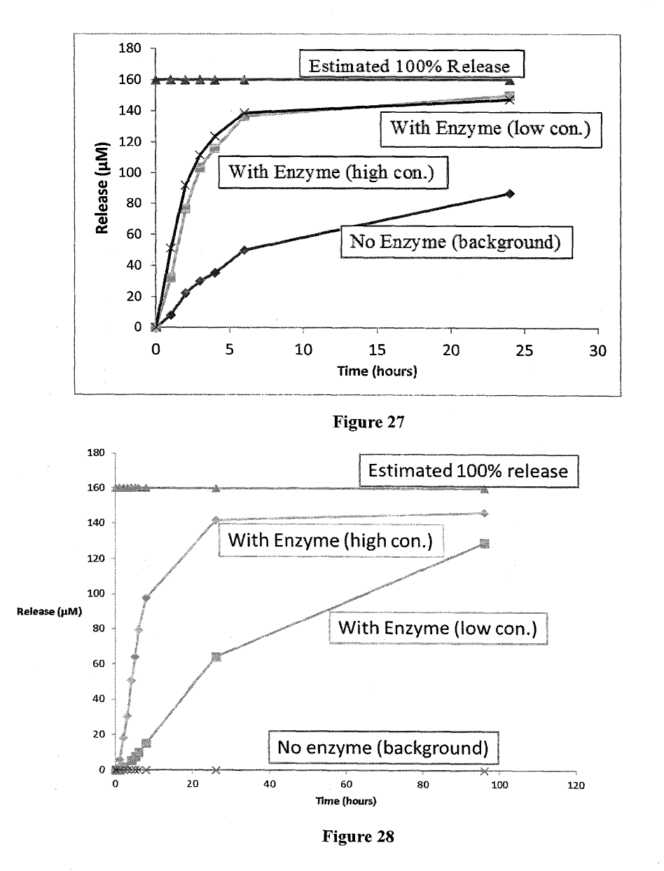

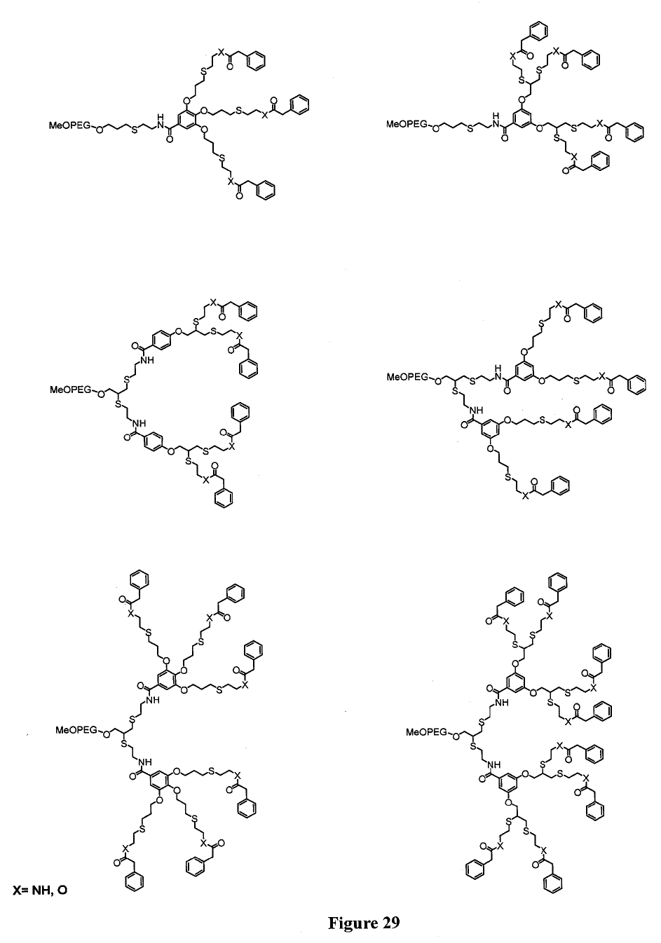

To evaluate the encapsulation ability of PLE responsive micelles, several encapsulation and release experiments were carried and the results are presented in FIGS. 27 and 28, which show the release of non-covalently encapsulated or covalently bound coumarin dyes from micelles based on hybrids 11b and 15b, respectively. In the case of micelles prepared from hybrid 11b, the hydrophobic fluorescence dye 7-diethylamino-3-carboxycoumarin butyl ester, was encapsulated non-covalently by dissolving the hydrophobic dye (160 μM) in a solution that contain micellar nanocarriers based on of hybrid 11b (40 μM). This solution was placed in a dialysis tube with a molecular weight cutoff (MWCO) of 1 kDa, which is expected to allow the escape of the dye molecule but to retain the polymeric hybrids. The dialysis tube was placed in an external tube filled with buffer solution and the amounts of released dyes were monitored by taking samples from the outer tube and analyzing them by HPLC. The results in FIG. 27, clearly indicate relatively high and efficient encapsulation of two dye molecules per polymer chain, as indicated by the amount of dye that was released in the absence of the activating enzyme. In the presence of the activating enzyme PLE, complete release of the dyes was observed in around 5 hours, demonstrating the great control over the enzymatically triggered disassembly and controlled release of the encapsulated dyes. Similar setup was used for micelles based on hybrid 15b. In this case, as the dyes are covalently attached at the core of the micellar nanocarriers, no background release was observed, indicating the high loading capacity (four dyes/polymer) and stability of the loaded micelles. Addition of low (0.23 μM) or high (8.5 μM) amounts of the enzyme PLE, resulted in the slow or fast release of the bound dyes, respectively (FIG. 28).

### Example 15

### Synthesis of Amphiphilic PEG-Dendron Hybrids

The hydrophobic end group may be conjugated to the hydroxy end-groups of the dendron through ester linkages (Scheme 4a). These esters can potentially be cleaved by enzymatic hydrolysis to release the parent active hydrophobic end group A.

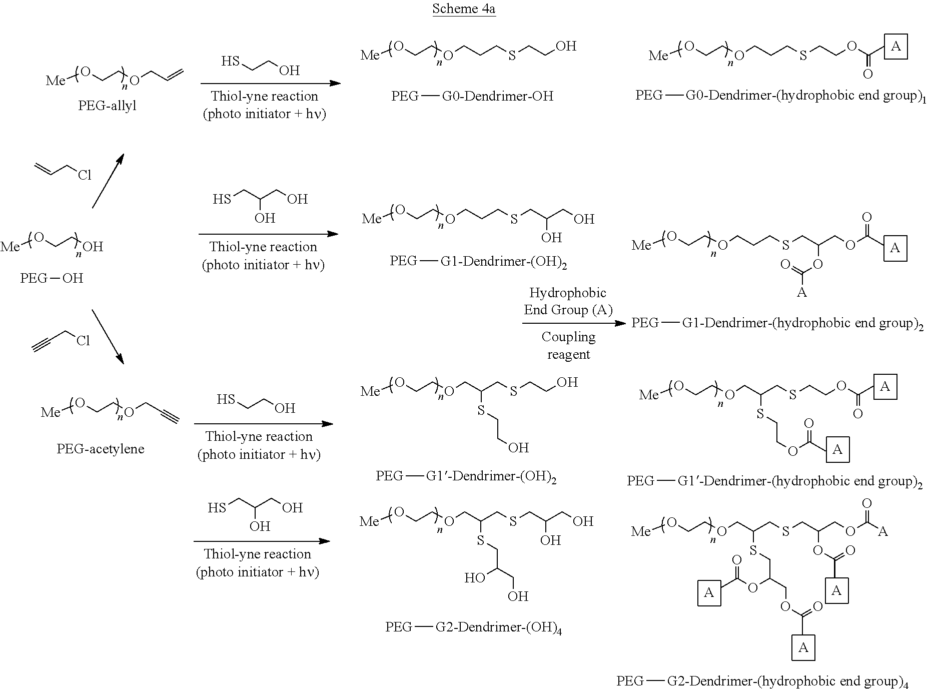

The utilization of ester linkages from either primary or secondary hydroxyls enables further control over the hydrolysis rate in addition to the length of the PEG and dendrons' generation. The synthesis of a second-generation dendron with primary ester linkages and a third-generation dendron are as illustrated in Scheme 4b.

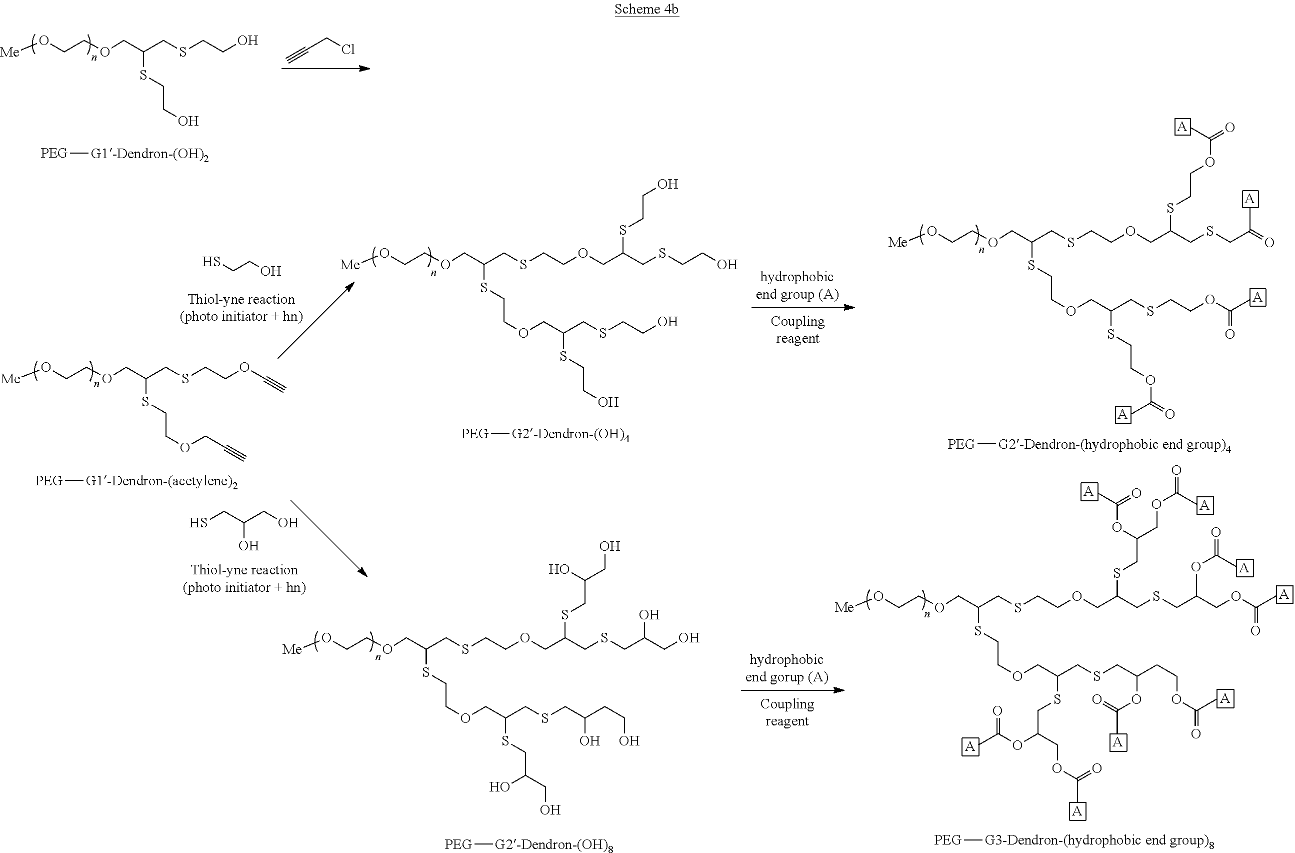

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-45. (canceled)

46. An amphiphilic hybrid delivery system in micellar form, comprising a hydrophilic polyethylene glycol (PEG) polymer conjugated to a hydrophobic dendron, the dendron comprising at least one enzymatically cleavable hydrophobic end group that is covalently attached to the dendron, wherein the micelle disassembles upon enzymatic cleavage of the hydrophobic end group.

47. The hybrid delivery system according to claim 46, wherein the enzymatically cleavable hydrophobic end group is or is derived from a biologically or diagnostically active agent, or wherein the enzymatically cleavable hydrophobic end group is biologically inactive, wherein the hydrophobic end group is released from the micelle upon enzymatic cleavage.

48. The hybrid delivery system according to claim 46, further comprising a biologically or diagnostically active compound encapsulated within said micelle, wherein said active compound is released upon disassembly of said micelle.

49. The hybrid delivery system according to claim 48, wherein the hydrophobic end group which is attached to said dendron and the compound which is encapsulated within said micelle are the same or are derived from the same compound, or wherein the hydrophobic end group which is attached to said dendron and the compound which is encapsulated within said micelle are different.

50. The hybrid delivery system according to claim 46, wherein the hydrophobic dendron comprises:
a first generation which is covalently bound to the PEG polymer, directly or through a linker or branching unit, and comprises at least one functional group capable of binding to a further generation or to said enzymatically cleavable hydrophobic end group; and
optionally, at least one additional generation which is covalently bound to said first generation or preceding generation, and optionally to a further generation, wherein each of said optional generations comprises at least one functional group capable of binding to said first generation, to a preceding generation, to a further generation, and/or to said enzymatically cleavable hydrophobic end group, each of said bonds being formed directly or through a linker or branching unit.

51. The hybrid delivery system according to claim 46, wherein each generation of the hydrophobic dendron comprises a linear or branched C1-C20 alkylene, C2-C20 alkenylene, C2-C20 alkynylene or arylene moiety which is substituted at each end with a group selected from the group consisting of —S—, —O—, —NH—, —C(═O)—, —C(═O)—O—, —O—C(═O)—O—, —C(═O)—NH—, —NH—C(═O)—NH—, NH—C(═O)—O—, —S(═O)—, —S(═O)—O—, PO(═O)—O—, and any combination thereof.

52. The hybrid delivery system according to claim 51, wherein each generation of the dendron is derived from a compound selected from the group consisting of HX—CH₂—CH₂—XH, HX—(CH₂)₁₋₃—CO₂H and HX—CH₂—CH(XH)—CH₂—XH wherein X is independently at each occurrence S, O or NH.

53. The hybrid delivery system according to claim 52, wherein each generation of the dendron is derived from a compound selected from the group consisting of HS—CH₂—CH₂—OH, HS—(CH₂)₁₋₃—CO₂H and HS—CH₂—CH(OH)—CH₂—OH.

54. The hybrid delivery system according to claim 46, further comprising a linker or branching unit which connects the PEG polymer to the first generation dendron and/or which forms a part of the first generation dendron, and/or which connects between dendron generations.

55. The hybrid delivery system according to claim 54, wherein the linker/branching unit moiety is selected from a group consisting of a substituted or unsubstituted acyclic, cyclic or aromatic hydrocarbon moiety, heterocyclic moiety, a heteroaromatic moiety or any combination thereof.

56. The hybrid delivery system according to claim 55, wherein the linker moiety/branching unit is a substituted arylene.

57. The hybrid delivery system according to claim 54, wherein the linker moiety/branching unit is connected to the PEG or to the dendron through a functional group selected from the group consisting of —O—, —S—, —NH—, —C(═O)—, —O—C(═O)—O—, —C(═O)—O—, —C(═O)—NH—, —NH—C(═O)—NH—, NH—C(═O)—O—, —S(═O)—, —S(═O)—O—, PO(═O)—O—, —C═C—, —C≡C—, —(CH₂)ₜ— wherein t is an integer of 1-10, and any combination thereof.

58. The hybrid delivery system according to claim 46, wherein the enzymatically cleavable hydrophobic end group is cleaved by an enzyme which is (i) present in greater amount at; or (ii) produced in greater quantity at, or (iii) has higher activity in cells near or at a site of disease or infection.

59. The hybrid delivery system according to claim 46, wherein the enzymatically cleavable hydrophobic end group is conjugated to the dendron through an enzymatically cleavable functional group selected from the group consisting of an ester, an amide, a carbamate, a carbonate, a urea, a sulfate, an amidine, an ether, a phosphate, a phosphoamide, sulfamates, and a trithionate.

60. The hybrid delivery system according to claim 59, wherein the enzymatically cleavable hydrophobic end group is conjugated to the dendron through an amide which is cleavable by an amidase,
wherein the amidase is preferably selected form the group of aryl-acylamidase, aminoacylase, alkylamidase, and phthalyl amidase.

61. The hybrid delivery system according to claim 59, wherein the enzymatically cleavable hydrophobic end group is conjugated to the dendron through an ester which is cleavable by an esterase,
wherein the esterase is preferably selected from the group consisting of carboxylesterase, arylesterase, and acetylesterase.

62. The hybrid delivery system according to claim 46, wherein the hydrophobic end group which is attached to the dendron is or is derived from a biologically or diagnostically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

63. The hybrid delivery system according to claim 62, wherein the compound encapsulated within said micelle is a biologically or diagnostically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

64. The hybrid delivery system according to claim 62, wherein the hydrophobic end group which is attached to the dendron and/or the compound which is encapsulated by said micelle are each independently selected from the group consisting of an anti-proliferative agent, a nonsteroidal anti-inflammatory agent, an antibiotic agent, an antimicrobial agent, an anti-viral agent, an immunosuppressant agent, an immunomodulator agent, an anti-hypertensive agent, a chemosensitizing agent, a anti-histamine agent, a general anesthetic agent, a local anesthetic agent, an analgesic agent, an anti-fungal agent, a vitamin, a fat-soluble vitamin, an hypnotic agent, a sedative agent, an anxiolytic agent, an antidepressant agent, an anticonvulsant agent, a narcotic analgesic agent, a narcotic antagonist agent, an anticholinesterase agent, a sympathomimetic agent, a parasympathomimetic agent, a ganglionic stimulating agent, a ganglionic blocking agent, an antimuscarinic agent, an adrenergic blocking agent, an autacoid and autacoid antagonist, digitalis and digitalis congeners, diuretic and saluretic agents, a cholesterol lowering agent, an antineoplastic agent, hemoglobin and hemoglobin derivatives and polymer, a hormonal agent, a hormonal antagonist agent, and combination thereof.

65. The hybrid delivery system according to claim 64, wherein the hydrophobic end group which is attached to the dendron and/or the compound which is encapsulated by said micelle are each independently selected from the group consisting of coumarin, methyl salicylate, aspirin, ibuprofen, naproxen, famciclovir, valacyclovir, acyclovir, penicillin-V, azlocillin, tetracycline, daunorubicin, doxorubicin, anthracycline, mitomycin C, aminopertin, mycophenolate mofetil, azathioprine, sirolimus, glucocorticoid, methotrexate, azathioprine, ciclosporin, tacrolimus, thalidomide, lenalidomide, pomalidomide, chlorothiazide, metolazone, amiloride, acrivastine, bilastine, buclizine, cimetidine, clobenpropit, desflurane, isoflurane, sevoflurane, propofol, methohexital, benzocaine, dibucaine, lidocaineproparacaine, paracetamol, morphine, oxycodone, celecoxib, flupirtine, amphotericin B, candicidin, bifonazole, butoconazole, fluconazole, abafungin, anidulafungin, retinol, thiamine, riboflavin, biotin, ergocalciferol, retinal, retinol, amobarbital, alprazolam, zopiclone, midazolam, amobarbital, alprazolam, sertraline, clobazam, codeine, naltrexone, physostigmine, ephedrine, dimethylphenylpiperazinium, pentamine, atropine, terazosin, histamine, hydrochlorothiazide, statin, tibolone, ganirelix acetate, septrin and derivatives thereof.

66. The hybrid delivery system according to claim 46, is represented by the structure of formula (I): 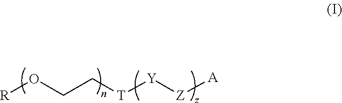
wherein
R is H or a C1-C4 alkylene group;
T is absent or is a functional group selected from the group consisting of —O—, —S—, —NH—, —C(═O)—, —O—C(═O)—O—, —C(═O)—O—, —C(═O)—NH—, —NH—C(═O)—NH—, NH—C(═O)—O—, —S(═O)—, —S(═O)—O—, PO(═O)—O—, —C═C—, —C≡C—, —(CH₂)ₜ— wherein t is an integer of 1-10, and any combination thereof.
Y is independently at each occurrence absent or is a linker moiety/branching unit;
Z is independently at each occurrence a dendron repeating unit selected from the group consisting of: 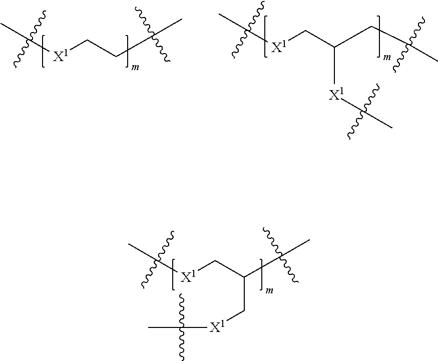
and any combination of the foregoing;
wherein X¹ is independently, at each occurrence, selected from the group consisting of a O, S and NH;
A is a hydrophobic end group which is conjugated to the dendron through an enzymatically cleavable functional group selected from the group consisting of an ester, an amide, a carbamate, a carbonate, a urea, a sulfate, an amidine, an ether, a phosphate, a phosphoamide, sulfamates, and a trithionate;
n is an integer in the range of 1 to 1,500, preferably 1 to 1,000; and
m and z are each an integer of 1 to 15.

67. The hybrid delivery system according to claim 66, wherein the hydrophobic end group A is or is derived from a biologically active agent selected from the group consisting of a pharmaceutically active agent, a cosmetic active agent, an anti-oxidant, a preservative, a vitamin, a coloring agent, a food additive, a fragrance, a hormone, an imaging agent, a diagnostic agent and an antibody.

68. The hybrid delivery system according to claim 66, wherein the terminal repeating unit of said dendron is represented by any of the following structures: 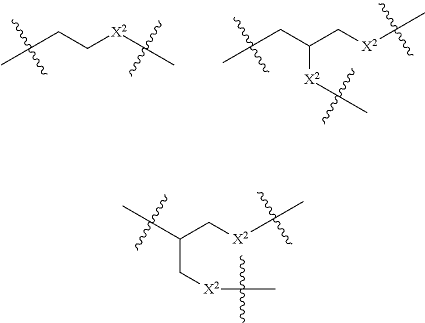
wherein X² has the same meaning as X¹.

69. The hybrid delivery system according to claim 66, wherein the hydrophobic end group A is conjugated to the dendron through an enzymatically cleavable functional group represented by the structure: 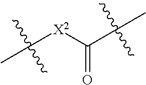
wherein X² is a part of the terminal repeating unit of said dendron and C(═O) is part of hydrophobic end group; or wherein X² is part of the hydrophobic end group and C(═O) is a part of the terminal repeating unit of said dendron, or wherein X²—C(═O) are part of the hydrophobic end group, or wherein X²—C(═O) is part of the terminal repeating unit of said dendron; and wherein X² has the same meaning as X¹.

70. The hybrid delivery system according to claim 66, which is represented by any one or more of the following structures: 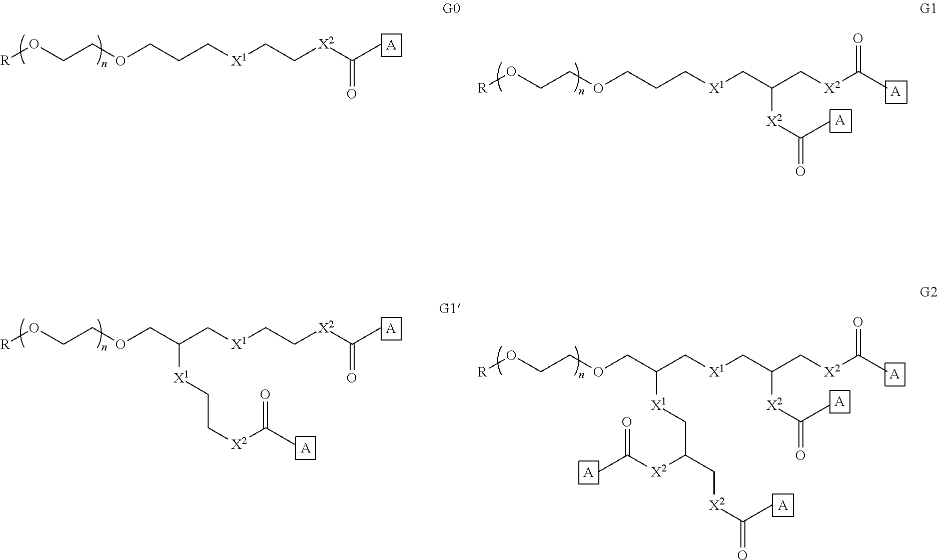 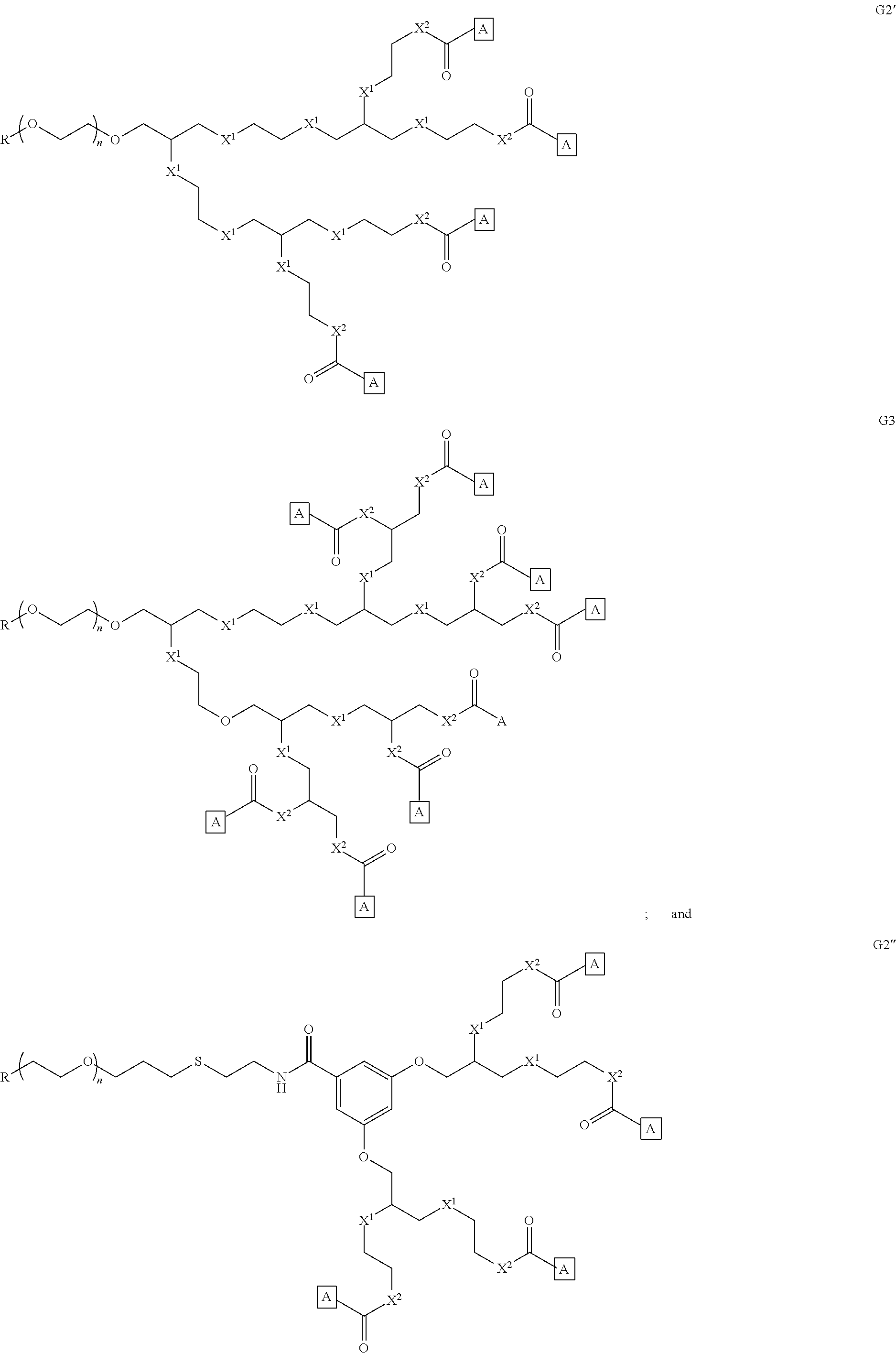
wherein each X¹ and X² is independently at each occurrence selected from the group consisting of O, S and NH; R is H or an C1-C4 alkylene group;
A, alone or together with C(═O) is a hydrophobic end group; and
n is an integer of 1 to 1,500, preferably 1 to 1,000.

71. The hybrid delivery system according to claim 66 which is represented by the structure of any of compounds 1a, 1b, 1c, 11a, 11b, 11c, 15a, 15b or 15c, or any of the structures of FIG. 29.

72. A method of delivering the amphiphilic hybrid system according to claim 46, comprising the step of contacting the amphiphilic hybrid delivery system with an enzyme to induce cleavage of the enzymatically cleavable hydrophobic end group, thereby disassembling said micelle.

73. A kit for delivering the amphiphilic hybrid system according to claim 46, comprising in one compartment the amphiphilic hybrid system, and in a second compartment an enzyme capable of cleaving the enzymatically cleavable hydrophobic end group so as to disassemble said micelle.
